SEQUENCE LISTING (5 sequences)

SEQ ID NO: 1 - DNA, 2208 bp, Homo sapiens
agctcccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacaccagtagtcccagctattcaggaggctgaggtgaggggatcacttaaggctgggaggttgaggctgcagtgagtcgtggttgcgccactgcagtccagcctgggcaacagtgagaccctgtctcaaaagccaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttcccttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgc

SEQ ID NO: 2 - DNA, 20 bp, synthetic primer
actccttcataaagccctcg

SEQ ID NO: 3 - DNA, 26 bp, synthetic primer
aactcgccgcacatctgaacttcagc

SEQ ID NO: 4 - DNA, 23 bp, synthetic primer
tcagcttggagttgatcccgtcg

SEQ ID NO: 5 - DNA, 26 bp, synthetic primer
aacaaacggcggattgaccgtaatgg

(19) United States
(12) Patent Application Publication
(10) Pub. No.: US 2007/0299029 A1
(43) Pub. Date: Dec. 27, 2007

(54) Hepatic stellate cell specific promoter and uses thereof

(75) Inventors: Lang Zhuo, Singapore (SG); Gunter Maubach, Singapore (SG); Chun-Yan Zhang, Singapore (SG)

Correspondence Address: KLARQUIST SPARKMAN, LLP, 121 SW SALMON STREET, SUITE 1600, PORTLAND, OR, 97204, US

(73) Assignee: Agency for Science, Technology and Research

(21) Appl. No.: 11/702,018

(22) Filed: Feb. 2, 2007

Related U.S. Application Data
(63) Continuation-in-part of application No. PCT/SG06/00149, filed on Jun. 9, 2006.
(60) Provisional application No. 60/688,733, filed on Jun. 9, 2005.
(60) Provisional application No. 60/764,762, filed on Feb. 3, 2006.

(51) Int. Cl. A61K 31/715; A61P 19/00; C12N 15/00; C12N 5/06; C12Q 1/68

(52) U.S. Cl. 514/44; 435/370; 435/455; 435/6

(57) ABSTRACT

Methods and reagents for effecting transgene expression in a hepatic stellate cell, isolated transgenic hepatic stellate cells, methods and reagents for identifying compounds with fibrogenesis modulating properties and uses thereof.

### CROSS REFERENCE TO RELATED APPLICATIONS

This application is a continuation-in-part application of international application PCT/SG2006/000149, filed Jun. 9, 2006, which claims benefit and priority from U.S. provisional patent application No. 60/688,733 filed Jun. 9, 2005, the contents of both of which are fully incorporated by reference. This application further claims benefit and priority from U.S. provisional patent application No. 60/764,762, filed Feb. 3, 2006, the contents of which is fully incorporated by reference.

### FIELD OF THE INVENTION

This invention relates generally to methods and reagents for effecting transgene expression in a hepatic stellate cell (HSC), transgenic HSCs and to methods and reagents for identifying compounds with fibrogenesis modulating properties.

### BACKGROUND OF THE INVENTION

Hepatic stellate cells (HSCs), previously known as Ito cells, lipocytes, perisinusoidal cells or fat-storing cells, are a minor cell type (roughly 5-8% of total liver cells) most commonly found in the sinusoidal area of adult livers. The basic pathobiology and history of HSC discovery have been reviewed elsewhere (Burt, (1999), J Gastroenterol. 34(3):299; Sato et al. (2003), Cell Struct Funct. 28(2):105). The major physiological functions of HSC include fat storage, vitamin A uptake and metabolism, and the production of extracellular matrix proteins. In the past decade, HSCs have been implicated in mounting a defense during hepatic injury, and mediating hepatic fibrogenesis by over-producing pro-fibrotic cytokines and consequently extracellular matrix (ECM) molecules. HSCs are believed to play a role in the pathogenesis of a number of clinically important conditions such as, for example, hepatic fibrosis, cirrhosis, portal hypertension and liver cancer (Geerts (2004), J. Hepatol. 40(2):331). Hence, HSCs have also become a target for the development of anti-fibrotic therapies (Bataller et al., (2001), Semin Liver Dis. 21(3):437; Bataller et al., (2005), J. Clin Invest. 115(2):209; Friedman (2003), J. Hepatol. 38 Suppl 1:S38).

Activation of HSCs is a dominant event in fibrogenesis. During activation, quiescent vitamin A storing cells are converted into proliferative, fibrogenic, proinflammatory and contractile ‘myofibroblasts’ (Friedman (2003), J. Hepatol. 38 Suppl 1:S38; Bataller et al. (2005), J. Clin Invest. 115(2):209; Cassiman et al. (2002), J. Hepatol. 36(2):200). HSC activation proceeds along a continuum that involves progressive changes in cellular function. In vivo, activated HSCs migrate and accumulate at the sites of tissue repair, secreting large amounts of ECM components and regulating ECM degradation (Cassiman et al. (2002), J. Hepatol. 36(2):200). HSC identity both in vitro and in vivo has been traditionally identified with antibodies. Initially, a polyclonal rabbit antibody against chicken desmin (an intermediate filament) was used to stain cells with stellate shape in liver slices and skeletal myofibrils in rat (Yokoi et al. (1984), Hepatology. 4:709). Additional antibodies against vimentin (another intermediate filament) and smooth muscle-alpha-actin (SMAA) were subsequently employed to study liver fibrosis in rat (Bhunchet et al. (1992), Hepatology. 16:1452; Baroni et al. (1996), Hepatology. 23(5):1189). Despite their poor tissue- and cell-specificity, these three markers (desmin, vimentin and SMAA) have remained a common battery for identifying HSCs. Glial fibrillary acidic protein (GFAP) has also been indicated to be a marker for HSCs (Buniatian et al. (1996), Eur J. Cell Biol. 70(1):23; Levy et al. (1999), Hepatology 29(6):1768; Cassiman et al., (2002), J. Hepatol. 36(2):200; Xu et al. (2005), Gut. 54:142).

Cell specific promoters are of great interest to those involved in genetic engineering for their potential to drive expression of a target gene in a specific subpopulation or subset of cells either in vitro or in vivo.

Several promoters have been investigated for their ability to express a gene of interest specifically in HSCs in vitro and in vivo. These promoters include the human collagen alpha 1 (ColI; Slack et al. (1991), Mol Cell Biol. 11(4):2066; Brenner et al. (1993), Hepatology 7(2):287; Yata et al. (2003), Hepatology 37:267), SMAA (Magness et al. (2004), Hepatology 40:1151), LIM domain protein CRP2 (CSRP2), tissue inhibitor of metalloproteinase-1 (TIMP-1) and smooth muscle-specific 22-kDa protein (SM22alpha) (Bahr et al. (1999), Hepatology 29(3):839; Herrmann et al. (2004), Liver International 24: 69).

In astrocytes, a fragment of the human GFAP (hGFAP) promoter has been shown to drive expression of operatively coupled transgenes in vitro and in vivo. The activity of this promoter fragment in non-astrocytic cells has been shown to be less predictable. The promoter fragment unreliably expressed lacZ in Müller cells in transgenic mice lines, leading to the suggestion that Müller cells may require regulatory elements beyond those contained in the promoter fragment (Brenner (1994), J. Neurosci. 14: 1030). In Schwann cells, the transcription initiation site of the endogenous GFAP promoter is 169 nucleotides upstream from the transcription initiator site in astrocytes (Feinsten et al. (1992) J. Neurosci Res. 32(1):1). Further, while Schwann cells are known to express endogenous GFAP, these cells are also unreliably labeled in hGFAP-LacZ transgenic mice (Zhuo (1997), Developmental Biology 187:36).

### SUMMARY OF THE INVENTION

The inventors here report that a 2.2 kb promoter fragment from the hGFAP gene may be used to drive transgene expression in HSCs, and further that this expression is upregulated in response to pro-fibrogenic factors. These results demonstrate that the 2.2 kb promoter from the hGFAP gene is not only capable of driving HSC-specific expression, but that the promoter contains additional regulatory sequences that are responsible for the induction of transgene transcription in HSCs.

There is thus provided a method for selectively expressing a transgenic product in HSC cells. The transgenic product may be used to identify HSCs or modulate hepatic fibrosis in vitro or in vivo.

A vector containing a marker molecule operably coupled to a GFAP promoter may be useful for identifying HSCs in vivo or in vitro. Transgenic HSCs comprising a transgene operably coupled to a GFAP promoter may be used in assays to study hepatic fibrogenesis, including the in vitro or in vivo screening of factors that may affect, reduce or inhibit fibrogenesis.

In one aspect, there is provided a method for expressing a transgenic product in a hepatic stellate cell, the method comprising transfecting the hepatic stellate cell with a vector comprising a glial fibrillary acidic protein promoter operably coupled to a DNA sequence encoding the transgenic product, wherein the glial fibrillary acidic protein promoter consists of the sequence set forth in SEQ. ID NO. 1, or is an allelic variant of the sequence set forth in SEQ ID NO: 1.

In another aspect there is provided an isolated transgenic hepatic stellate cell, the cell comprising a transgene operably coupled to a glial fibrillary acidic protein promoter, wherein the promoter consists of the sequence set forth in SEQ ID NO: 1, or a sequence that is an allelic variant of SEQ ID NO: 1.

In another aspect, there is provided a method of identifying a fibrogenesis modulating agent, the method comprising: detecting a first expression level of a gene that is operably coupled to a glial fibrillary acidic protein promoter in an expression system in the absence of a test compound; detecting a second expression level of the gene in the expression system in the presence of the test compound; and comparing the first expression level and the second expression level; whereby the first expression level greater than the second expression level indicates that the test compound is an anti-fibrotic agent and the first expression level less than the second expression level indicates that the test compound is a pro-fibrotic agent, and wherein the promoter consists of the sequence set forth in SEQ ID NO: 1, or a sequence that is an allelic variant of SEQ ID NO: 1.

In another aspect there is provided a use of a vector comprising a DNA sequence encoding a therapeutic product operably coupled to a glial fibrillary acidic protein promoter, wherein the glial fibrillary acidic protein promoter consists of the sequence set forth in SEQ. ID NO. 1, or is an allelic variant of the sequence set forth in SEQ ID NO: 1 for treating a hepatic fibrosis related disorder.

In another aspect, there is provided a use of a vector comprising a DNA sequence encoding a therapeutic product operably coupled to a glial fibrillary acidic protein promoter, wherein the glial fibrillary acidic protein promoter consists of the sequence set forth in SEQ. ID NO. 1, or is an allelic variant of the sequence set forth in SEQ ID NO: 1 for the preparation of a medicament for treating a hepatic fibrosis related disorder.

In another aspect, there is provided a pharmaceutical preparation comprising a vector comprising a sequence encoding a therapeutic product operably coupled to a glial fibrillary acidic protein promoter, wherein the glial fibrillary acidic protein promoter consists of the sequence set forth in SEQ. ID NO. 1, or is an allelic variant of the sequence set forth in SEQ ID NO: 1 for treating a hepatic fibrosis related disorder and a physiological carrier.

In yet another aspect there is provided a method of treating a hepatic fibrosis related disorder in a subject, the method comprising administering to the subject an effective amount of a transgenic HSC, wherein the transgenic HSC comprises a transgene encoding a therapeutic product, said transgene operably coupled to a glial fibrillary acidic protein promoter, and wherein said promoter consists of the sequence set forth in SEQ. ID NO. 1, or is an allelic variant of the sequence set forth in SEQ ID NO: 1.

In another aspect, there is provided a method of diagnosing the presence of hepatic fibrosis in a subject or determining the prognosis of a subject having or being likely to develop hepatic fibrosis, the method comprising: detecting expression level of a first gene that is operably coupled to a glial fibrillary acidic protein promoter in a hepatic stellate cell from the subject; and comparing the expression level in the hepatic stellate cell from the subject with expression of a second gene that is operably coupled to a glial fibrillary acidic protein promoter in a hepatic stellate cell that is not associated with hepatic fibrosis; wherein the glial fibrillary acidic protein promoter consists of the sequence set forth in SEQ ID NO: 1, or a sequence that is an allelic variant of SEQ ID NO: 1.

In still yet another aspect, there is provided a kit comprising a vector comprising a sequence encoding a therapeutic product operably coupled to a glial fibrillary acidic protein promoter, wherein the glial fibrillary acidic protein promoter consists of the sequence set forth in SEQ. ID NO. 1, or is an allelic variant of the sequence set forth in SEQ ID NO. 1, and instructions for treating a hepatic fibrosis related disorder in a subject.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate by way of example only, embodiments of the present invention:

FIG. 1 depicts a map of the pcdna4/GFAP2.2-Lac Z vector;

FIG. 2 depicts β-galactosidase staining in C3A, Hep2G and NIH/3T3 cells after transient transfection with vectors containing the lacZ gene under the control of a CMV promoter (top) or a GFAP promoter (bottom);

FIG. 3 depicts gel electrophoresis of PCR products from pcDNA4/GFAP2.2-LacZ transfected (lanes 3, 5, 7) and non-transfected (lanes 2, 4, 6) T6 cells (lane 2 and 3), C6 cells (lanes 4 and 5) and HeLa cells (lanes 6 and 7). Panel A depicts a GFAP-lacZ PCR products from genomic DNA. Panel B depicts LacZ RT-PCR products from 50 ng total RNA. Panel C depicts β-actin RT-PCR products. In each panel lane 1 is a 1 kb DNA ladder;

FIG. 4 depicts the 3-galactosidase staining in T6, C6 and HeLa stably transfected with the 2.2 kb hGFAP-lacZ transgene;

FIG. 5 depicts B-galactosidase staining in transfected T6 cells incubated with different concentrations of TGF-β1. The T6/lacZ/C1 cells were incubated with the indicated concentrations of TGF-β1 for three days prior to staining;

FIG. 6 depicts β-galactosidase staining in transfected T6 cells incubated with different concentrations of PDGF-BB. The T6/lacZ/C1 cells were incubated with the indicated concentrations of PDGF-BB for three days prior to staining;

FIG. 7 depicts β-galactosidase staining in transfected T6 cells incubated with different concentrations of LPS. The T6/lacZ/C1 cells were incubated with the indicated concentrations of PDGF-BB for three days prior to staining;

FIG. 8 depicts the β-galactosidase activity of T6/lacZ/C1 cells incubated with 1 ng/ml of the TGF-β1 as a function of incubation time. The control on the left shows the activity for the T6 stable transfected with pcDNA4/GFAP2.2-LacZ under normal growth conditions (37° C., 5% CO₂, 10% serum). The first data point represents the cells harvested 12 hours after changing the media to 0.5% serum. Data given as mean ±SEM. +p<0.001 compared with the control; * p<0.05, ** p<˜0.005, *** p<0.001 compared with the non-induced sample;

FIG. 9 depicts the time and TGF-β1 concentration dependent 3-galactosidase activity of T6/lacZ1/C1 cells stably transfected with pcDNA4/GFAP2.2-LacZ. The samples were assayed for different concentrations of TGF-β1 at different time points (24, 48, 72 hours). The four columns at each time point represent the four different concentrations 0, 0.1, 1, 10 ng/ml TGF-β1 (from left to right). Data of six experiments as mean ±SEM. * P<0.05, ** p<0.001 compared with non-induced sample;

FIG. 10 depicts the inducing effect of 10 ng/ml of TGF-β1 on the fold induction of β-galactosidase specific activity under control of the GFAP 2.2 kb promoter fragment. Each induced sample was compared with its respective non-induced sample from the same experiment. The data represent the mean ±SEM of six experiments. * p<0.001 compared with non-induced sample;

FIG. 11 demonstrates that 1 ng/ml TGF-β1 induces endogenous GFAP mRNA expression in T6/LacZ/C1 cells. *P<0.01, **P<0.005, ***P<0.001, compared with control;

FIG. 12 illustrates that the 2.2 kb hGFAP-GFP transgene was expressed specifically in the hepatic stellate cells in the liver of a transgenic mouse model. A). Liver tissue section stained with anti-GFP antibody; B) The same tissue section stained with anti-GFAP antibody; C) Merged image of A) and B); D) Light transmission of the same tissue section;

FIG. 13 depicts the chemical structures of EGCG and genistein;

FIG. 14 illustrates that EGCG and genistein suppressed proliferation of the T6/GFAP-lacZ cells. Cells were grown in full medium to 70-80% confluence in 96-well plates, followed by compound treatment of various concentrations (0-40 μM) in a low serum medium for 48 hours before assaying for proliferation. Signal was normalized against vehicle treatment (0 μM compound). Data was obtained from three independent experiments and presented as mean ±SE. IC50 were 31 μM for EGCG and 25 μM for genistein respectively;

FIG. 15 depicts X-gal staining of T6/GFAP-lacZ cells. Incubation with A) DMSO vehicle control; B) 25 μM EGCG; and C) 40 μM genistein in a low serum DMEM for 24 hours. Images were captured after 60 min of X-gal staining;

FIG. 16A depicts time- and dose-dependent suppression of GFAP-lacZ by EGCG. The β-galactosidase activity was assayed for the T6/GFAP-lacZ cells treated six doses of EGCG (0, 0.1325, 1.25, 5, 10 and 20 μM) at different time points (2, 4, and 24 hours). Data were presented as mean ±SE (N=6, * P<0.05, ** p<0.001), when compared to the non-suppressed control (0 μM);

FIG. 16B depicts time-dependent suppression of GFAP mRNA by EGCG. The T6/GFAP-LacZ cells were incubated with 10 μM of EGCG in a low serum DMEM for various times (0, 8, 16, 24 and 48 hours), and the endogenous rat GFAP mRNA was quantified with real-time RT-PCR. Data were presented as mean ±SE, N=6, *P<0.05, **P<0.01, when compared to the treatment at 0 hour;

FIG. 16C depicts dose-dependent suppression of GFAP mRNA by EGCG. The T6/GFAP-LacZ cells were treated with various doses of EGCG (0, 0.1325, 1.25, 5, 10, and 20 μM) in a low serum DMEM for 48 hours, and the endogenous rat GFAP mRNA was quantified with real-time RT-PCR. Data were presented as mean ±SE, N=6, *P<0.001, when compared to treatment with 0 μM;

FIG. 17A depicts attenuation of TGF-β1-induced GFAP-lacZ elevation by EGCG. The T6/GFAP-LacZ cells were treated with 10 ng/ml TGF-β1 for 24 hours (left column); with 10 ng/ml TGF-β1 for 24 hours followed by 20 μM of EGCG for additional 2 hours (middle column); and with 20 μM of EGCG and 10 ng/ml TGF-β1 for 26 hours (right column). Data were presented as mean ±SE, N=6, * P<0.005, when compared to 10 ng/ml TGF-β1 treated alone control (left column);

FIG. 17B depicts attenuation of TGF-β1-induced GFAP mRNA elevation by EGCG. The T6/GFAP-LacZ cells were treated with 1 ng/ml TGF-β1 for 48 hours (left column); with 1 ng/ml TGF-β1 for 48 hours followed by 5 μM of EGCG for additional 8 hours (middle column); and with 5 μM of EGCG and 1 ng/ml TGF-β1 for 56 hours (right column). Data were presented as mean ±SE, N=6, * P<0.001, when compared to 1 ng/ml TGF-β1 treated alone control (left column);

FIG. 18A depicts attenuation of PDGF-induced GFAP-lacZ activation by EGCG. The T6/GFAP-LacZ cells were subject to different treatments and β-galactosidase activity was assayed. Starting from left to right, column 1: vehicle control; column 2: 10 ng/ml PDGF-BB for 48 hours; column 3: 10 ng/ml PDGF for 48 hours followed by 20 μM of EGCG for additional 2 hours; and column 4: 20 μM of EGCG and 10 ng/ml PDGF for 50 hours. Data were presented as mean ±SE, N=6, * P<0.01, **P<0.001;

FIG. 18B depicts attenuation of PDGF-induced GFAP mRNA expression. The T6/GFAP-LacZ cells were subject to different treatments and mRNA was quantified by real-time RT-PCR. Starting from left to right, column 1: vehicle control; column 2: 10 ng/ml PDGF for 24 hours; column 3: 10 ng/ml PDGF for 24 hours followed by 10 μM of EGCG for additional 24 hours; and column 4: 10 μM of EGCG and 10 ng/ml PDGF for 48 hours. Data were presented as mean ±SE, N=6, * P<0.05, **P<0.001;

FIG. 19A depicts suppression of transgenic GFAP-lacZ by genistein. The T6/GFAP-LacZ cells were treated with different concentrations of genistein and assayed for β-galactosidase activity at various times (4, 8, 24, and 48 hours). The six columns (from left to right) represent six different concentrations of genistein (0, 0.1325, 1.25, 5, 10, 20, and 40 μM) Data were presented as mean ±SE, N=6, * P<0.05, ** P<0.001, when compared to treatment with 0 μM of genistein;

FIG. 19B depicts time-dependent suppression of GFAP mRNA by genistein. The T6/GFAP-lacZ cells were incubated with 10 or 20 μM of genistein in a low serum DMEM for various times (0, 8, 16, 24, 48, and 72 hours). The endogenous level of rat GFAP mRNA was quantified with real-time RT-PCR. Data were presented as mean ±SE, N=6, *P<0.05, **P<0.001, when compared to treatment at 0 hour;

FIG. 19C depicts dose-dependent suppression of GFAP mRNA by genistein. The T6/GFAP-lacZ cells were incubated in a low serum DMEM containing various concentrations (0, 0.1325, 1.25, 5, 10, 20, and 40 mM) of genistein for 48 hours, and the endogenous rat GFAP mRNA was quantified with real-time RT-PCR. Data were presented as mean ±SE, N=6, *P<0.001, when compared to vehicle treatment

FIG. 20A depicts attenuation of TGF-β1-induced T6/GFAP-lacZ activation by genistein. The T6/GFAP-lacZ cells were subject to different treatments, left column: 10 ng/ml TGF-β1 for 24 hours; middle column: 10 ng/ml TGF-β1 for 24 hours followed by 40 μM of genistein for additional 24 hours; and right column: 40 μM genistein and 10 ng/ml TGF-β1 for 48 hours, and the β-galactosidase activity was assayed. Data were presented as mean ±SE, N=6, * P<0.005 when compared to 10 ng/ml TGF-β1 treated alone (left column);

FIG. 20B depicts attenuation of TGF-1-induced GFAP mRNA by genistein. The T6/GFAP-lacZ cells were subject to different treatments and GFAP mRNA level was quantified with real-time RT-PCR. Left column: 1 ng/ml TGF-β1 for 48 hours; Middle column: 1 ng/ml TGF-β1 for 48 hours followed by 20 μM of genistein for additional 8 hours; and Left column: 20 μM of genistein and 1 ng/ml TGF-β1 for 56 hours. Data were presented as mean ±SE, N=6, * P<0.001, when compared to 1 ng/ml TGF-β1 treated alone sample (left column);

FIG. 21A depicts attenuation of PDGF-BB-induced T6/GFAP-lacZ activation by genistein. The T6/GFAP-lacZ cells were subject to different treatments, starting from left to right, column 1: vehicle control; column 2: 10 ng/ml PDGF-BB for 48 hours; column 3: 10 ng/ml PDGF-BB for 48 hours followed by 40 μM of genistein for additional 24 hours; and column 4: 40 μM of genistein and 10 ng/ml PDGF-BB for 72 hours. Data were presented as mean ±SE, N=6, * P<0.005, **P<0.001; and

FIG. 21B depicts attenuation of PDGF-BB-induced GFAP mRNA elevation by genistein. The T6/GFAP-lacZ cells were subject to different treatments, and the endogenous GFAP mRNA level was quantified with real-time RT-PCR. Starting from to right, column 1: vehicle control; column 2: 10 ng/ml PDGF for 24 hours; column 3: 10 ng/ml PDGF for 24 hours followed by 20 μM of genistein for additional 24 hours, and column 4: 20 μM of genistein and 10 ng/ml PDGF for 48 hours. Data were presented as mean ±SE, N=6, * P<0.005, **P<0.001.

### DETAILED DESCRIPTION

The inventors have surprisingly discovered that a fragment of the human GFAP promoter is active in HSCs. More specifically, a 2.2 kb fragment corresponding to nucleotides −2163 to +47 (SEQ ID NO: 1) of the hGFAP gene (Accession Number M67446) is capable of driving transgene expression in rat T6 HSC cells, but not in HepG2 hepatocytes, C3A cells (a clonal derivative of HepG2) or HeLa cells. In HSC cell culture, the expression of a transgene operably coupled to an hGFAP promoter is induced in a dose-dependent manner by the pro-fibrotic factors transforming growth factor-β1 (TGF-β1), platelet derived growth factor-BB (PDGF-BB) and lipopolysaccharide (LPS).

There is presently provided a method for effecting HSC-specific transgene expression, the method comprising transfecting an HSC cell with a vector comprising a transgene operably coupled to a GFAP promoter.

As used herein, “HSC” includes any hepatic stellate cell, including isolated HSCs, as well as transgenic HSCs as described herein in an in vivo context, for example an animal model comprising a transgenic HSC as described herein, unless otherwise specified. HSC also includes endogenous HSCs in an in vivo context.

Unless the context dictates otherwise, as used herein “HSC” includes both a quiescent and activated HSCs. Activated HSCs may be obtained by known methods, such as, for example, by culturing primary HSCs on uncoated plastic substrates.

In particular embodiments, “HSC” includes a primary hepatic stellate cell (or cells) isolated from liver, as well as cells derived from the in vitro passage of primary HSCs. Methods for isolating primary HSCs would be known to a person skilled in the art, for example, those described in Friedman (1992), Hepatology 12:3234 and Cassiman (1999), Am. J. Pathol. 155(6):1831).

While primary HSCs may be isolated from liver, this approach is generally limited by the low yield of HSCs, frequent presence of other cell types and the low (i.e. <1%) transfection efficiency of primary HSCs (Xu et al. (2005), Gut. 54:142). As used herein, “HSC” also includes model HSC-derived cell or cells, such as, for example, the immortalized rat HSC-T6 cell. Rat HSC-T6 cells exhibit an activated phenotype reflected in their fibroblast-like shape, rapid proliferation in culture and the expression of desmin, SMAA, GFAP and vimentin (Vogel et al. (2000), J Lipid Res. 41(6):882). Other HSC-derived model cell lines would be known to a person skilled in the art and include, for example, the human LX-1 or, more preferably, the LX-2 cell lines (Xu et al. (2005), Gut. 54:142). Both LX-1 and LX-2 cell lines express a number of markers of activated HSC, including SMAA and GFAP, and the LX-2 line has been shown to possess a transfection efficiency exceeding 30%. HSC-T6, LX-1 and LX-2 cells may be deactivated by growth in Matrigel™ or by culture in low serum media (Xu et al. (2005), Gut. 54:142). In a specific embodiment, the HSC is HSC-T6.

As used herein, “expression” or “expressing” refers to production of any detectable level of the transcription product of a gene, including a transgene, in an HSC. As will be understood by a person skilled in the art, transcription levels may be determined by direct methods that measure the amount of gene or transgene mRNA, for example, Northern blot, quantitative RT-PCR, RNA run-on assays, gene chip analysis. Alternatively, transgene expression may be measured indirectly by measuring the optical, colorimetric, fluorogenic, immunogenic or enzymatic properties of the protein product translated from the transgene mRNA. For example, the activity of a reporter gene, such as β-galactosidase, may be determined by known assays using the chromogenic substrate 5-Bromo-4-Chloro-3-Indolyl-BD-Galactopyranoside (X-gal). Alternatively, transgene expression may be determined by known immunological methods, for example, Western analysis.

As used herein, “transgene” refers to an exogenous DNA coding sequence. The transgene may encode an RNA product, including an mRNA that can be translated into a protein or polypeptide product in the transfected cell. The coding region of the transgene may be contiguous or may contain one or more introns. As would be understood by a person skilled in the art, if the transgene is derived from bacteria or viral sources, the codon preference of the transgene may be optimized to that of the target HSC.

A “transgenic product” is therefore a product expressed from the transgene or a product that results from transgene expression, including an RNA product or a translated protein product. Thus, a transgene encodes a transgenic product, including RNA transgenic products and protein transgenic products.

As used herein, “transfecting” refers to any process wherein exogenous nucleic acids are introduced into a HSC, and includes viral and non-viral methods. Examples of viral methods would be known to a person skilled in the art and include, for example, the administration of lentiviral vectors. Non-viral transfection methods would also be known to the skilled person and include, for example, naked plasmids, DEAE-dextran, calcium phosphate co-precipitation, microinjection, electroporation, nucleofection (Amaxa), liposome-mediated transfection, non-liposomal lipid preparations, cationic lipids, and polycationic polymers. Many of the non-viral transfection reagents and protocols are commercially available and would be known to a person skilled in the art. In specific embodiments, transfection may be effected by Lipofectamine™ 2000 (Invitrogen) according to the directions provided by the manufacturer. In other embodiments, transfection may be effected by FuGene™ 6 (Roche) according to the directions provided by the manufacturer.

A person skilled in the art would know how to identify a transfected HSC. For example, where the transfected nucleic acid encodes a selectable marker conferring cellular resistance against a drug, transfectants may be identified by exposing cells to that drug. For example, cells transfected with a nucleic acid encoding the Zeo® selectable marker may be identified by exposing the cells to culture media containing Zeocin™. Stable transgenic HSC lines may be selected by increasing the concentration of the drug and maintaining the transfected cells at the higher drug concentrations for an appropriate period of time, which will depend, among other things, on the proliferative rate of the cells. In some instances, the stable transfected cell lines may be obtained after at least 6 weeks of drug selection.

Alternatively, where the transfected HSC expresses a fluorescent marker protein, for example, GFP or any of its fluorogenic derivatives, transfected cells may be selected by optical methods, such as, for example, fluorescent activated cell sorting (FACS) (Yata et al. (2003), Hepatology. 37:267). Other methods of identifying transfectants based on nucleic acid hybridization, such as, for example, southern analysis and/or PCR amplification, would be known to a person skilled in the art.

A first nucleic acid sequence is operably coupled with a second nucleic acid sequence when the sequences are placed in a functional relationship. For example, a coding sequence is operably coupled to a promoter if the promoter activates transcription of the coding sequence. Similarly, a promoter and an enhancer are operably coupled when the enhancer increases the transcription of operably coupled sequences. Enhancers may function when separated from promoters and as such, an enhancer may be operably coupled to a promoter even though it is not contiguous to the promoter. Generally, however, operably coupled sequences are contiguous.

As would be understood by a person skilled in the art, the GFAP promoter sequence and the operably coupled transgene may be contained within a larger DNA vector. The vector may contain additional elements that allow for the integration, selection, replication or manipulation of the vector. For example, the vector may contain a selection marker, such as, for example, the neo and Zeo® genes, which may confer cellular resistance to G-418 and Zeocin™, respectively. Other selection markers would be known to a person skilled in art. Other additional elements would also be known to a person skilled in the art and include, for example, a multiple cloning site (MCS), and a transcription termination sequence, such as, for example, bovine growth hormone polyadenylation signal sequence (BGH pA).

The vector may be linear or circular, and, if circular, may be supercoiled. As would be known to a person skilled in the art, the efficiency of chromosomal integration may be enhanced by providing a linear vector, whereas episomal transfection may be more efficient with supercoiled DNA. Linear vectors may be prepared from circular vectors by known methods, for example, by cutting with an appropriate restriction endonuclease.

In one embodiment, the GFAP promoter is comprised of a 2.2 kb fragment corresponding to nucleotides −2163 to +47 of the hGFAP gene (Besnard et al. (1991), J Biol Chem. 266(28):18877; Brenner et al. (1994), J. Neurosci. 14: 1030; Zhuo et al. (1997), Developmental Biology 187:36). As detailed more fully in the accompanying examples, this 2.2 kb hGFAP promoter fragment can direct transgene expression in HSCs (but not in HepG2 or HeLa cells), and is induced by known fibrogenic factors.

In a specific embodiment, the 2.2 kb hGFAP promoter fragment has the following sequence [SEQ ID NO: 1]:

GAGCTCCCACCTCCCTCTCTGTGCTGGGACTCACAGAGGGAGACCTCAGG
AGGCAGTCTGTCCATCACATGTCCAAATGCAGAGCATACCCTGGGCTGGG
CGCAGTGGCGCACAACTGTAATTCCAGCACTTTGGGAGGCTGATGTGGAA
GGATCACTTGAGCCCAGAAGTTCTAGACCAGCCTGGGCAACATGGCAAGA
CCCTATCTCTACAAAAAAAGTTAAAAAATCAGCCACGTGTGGTGACACAC
ACCAGTAGTCCCAGCTATTCAGGAGGCTGAGGTGAGGGGATCACTTAAGG
CTGGGAGGTTGAGGCTGCAGTGAGTCGTGGTTGCGCCACTGCAGTCCAGC
CTGGGCAACAGTGAGACCCTGTCTCAAAAGCCAAAAAAAAAAAAAAAAAA
AAAAAGAACATATCCTGGTGTGGAGTAGGGGACGCTGCTCTGACAGAGGC
TCGGGGGCCTGAGCTGGCTCTGTGAGCTGGGGAGGAGGCAGACAGCCAGG
CCTTGTCTGCAAGCAGACCTGGCAGCATTGGGCTGGCCGCCCCCCAGGGC
CTCCTCTTCATGCCCAGTGAATGACTCACCTTGGCACAGACACAATGTTC
GGGGTGGGCACAGTGCCTGCTTCCCGCCGCACCCCAGCCCCCCTCAAATG
CCTTCCGAGAAGCCCATTGAGCAGGGGGCTTGCATTGCACCCCAGCCTGA
CAGCCTGGCATCTTGGGATAAAAGCAGCACAGCCCCCTAGGGGCTGCCCT
TGCTGTGTGGCGCCACCGGCGGTGGAGAACAAGGCTCTATTCAGCCTGTG
CCCAGGAAAGGGGATCAGGGGATGCCCAGGCATGGACAGTGGGTGGCAGG
GGGGGAGAGGAGGGCTGTCTGCTTCCCAGAAGTCCAAGGACACAAATGGG
TGAGGGGACTGGGCAGGGTTCTGACCCTGTGGGACCAGAGTGGAGGGCGT
AGATGGACCTGAAGTCTCCAGGGACAACAGGGCCCAGGTCTCAGGCTCCT
AGTTGGGCCCAGTGGCTCCAGCGTTTCCAAACCCATCCATCCCCAGAGGT
TCTTCCCATCTCTCCAGGCTGATGTGTGGGAACTCGAGGAAATAAATCTC
CAGTGGGAGACGGAGGGGTGGCCAGGGAAACGGGGCGCTGCAGGAATAAA
GACGAGCCAGCACAGCCAGCTCATGTGTAACGGCTTTGTGGAGCTGTCAA
GGCCTGGTCTCTGGGAGAGAGGCACAGGGAGGCCAGACAAGGAAGGGGTG
ACCTGGAGGGACAGATCCAGGGGCTAAAGTCCTGATAAGGCAAGAGAGTG
CCGGCCCCCTCTTGCCCTATCAGGACCTCCACTGCCACATAGAGGCCATG
ATTGACCCTTAGACAAAGGGCTGGTGTCCAATCCCAGCCCCCAGCCCCAG
AACTCCAGGGAATGAATGGGCAGAGAGCAGGAATGTGGGACATCTGTGTT
CAAGGGAAGGACTCCAGGAGTCTGCTGGGAATGAGGCCTAGTAGGAAATG
AGGTGGCCCTTGAGGGTACAGAACAGGTTCATTCTTCGCCAAATTCCCAG
CACCTTGCAGGCACTTACAGCTGAGTGAGATAATGCCTGGGTTATGAAAT
CAAAAAGTTGGAAAGCAGGTCAGAGGTCATCTGGTACAGCCCTTCCTTCC
CTTTTTTTTTTTTTTTTTTGTGAGACAAGGTCTCTCTCTGTTGCCCAGGC
TGGAGTGGCGCAAACACAGCTCACTGCAGCCTCAACCTACTGGGCTCAAG
CAATCCTCCAGCCTCAGCCTCCCAAAGTGCTGGGATTACAAGCATGAGCC
ACCCCACTCAGCCCTTTCCTTCCTTTTTAATTGATGCATAATAATTGTAA
GTATTCATCATGGTCCAACCAACCCTTTCTTGACCCACCTTCCTAGAGAG
AGGGTCCTCTTGCTTCAGCGGTCAGGGCCCCAGACCCATGGTCTGGCTCC
AGGTACCACCTGCCTCATGCAGGAGTTGGCGTGCCCAGGAAGCTCTGCCT
CTGGGCACAGTGACCTCAGTGGGGTGAGGGGAGCTCTCCCCATAGCTGGG
CTGCGGCCCAACCCCACCCCCTCAGGCTATGCCAGGGGGTGTTGCCAGGG
GCACCCGGGCATCGCCAGTCTAGCCCACTCCTTCATAAAGCCCTCGCATC
CCAGGAGCGAGCAGAGCCAGAGCAGGTTGGAGAGGAGACGCATCACCTCC
GCTGCTCGC.

In other embodiments, the GFAP promoter is any naturally occurring or engineered sequence that is an allelic variant or derivative of the 2.2 kb hGFAP sequence. As used herein, allelic variants or derivatives contemplate sequences that contain one or more nucleotide additions, substitutions and deletions while retaining the ability to direct selective transgene expression in HSC cells. For example, in different embodiments, the GFAP promoter may correspond to smaller fragments of the 2.2 kb hGFAP promoter that retain the ability to direct HSC-specific expression. Allelic variants of the hGFAP promoter also include naturally occurring homologous sequences from other organisms or allelic variants or derivatives thereof that retain the ability to direct HSC-selective transgene expression. Methods for identifying allelic variants or derivatives directing HSC-specific expression would be known to a person skilled in the art, for example, by deletion mapping.

Allelic variants of the GFAP promoter include any GFAP promoter that normally drives expression of an endogenous glial fibrillary acidic protein when found in the natural occurring context within a cell. For example, an allelic variant of the GFAP promoter may be a promoter that is naturally found operably coupled to the coding sequences defined by Accession No. NM002055, Accession No. AK098380, Accession No. BC041765 or Accession No. J04569, and which drives expression of such a coding region in the endogenous cellular context. Allelic variants of the GFAP promoter may be naturally occurring allelic variants of the sequence defined by Accession No. M67446.

In still other embodiments, the GFAP promoter is a recombinant hybrid promoter comprising one or more heterologous enhancer elements operably coupled to all or a portion of the 2.2 kb hGFAP promoter sequence, or an allelic variant or derivative thereof, provided the hybrid promoter is capable of selectively directing expression in a HSC. In this context, selective expression refers to a promoter that can direct the expression of an operably coupled transgene in HSCs but not in non-HSC liver cells such as, for example, hepatocytes.

The enhancer elements in the hybrid promoter may be selected from known elements, such as, for example, enhancer elements from the human cytomegalovirus, or may be novel elements identified thorough known methods, such as, for example, enhancer trap assays.

Hybrid promoters may be synthesized using standard molecular biology and molecular cloning techniques known in the art, for example, as described in Sambrook et al. (2001) Molecular Cloning: a Laboratory Manual, 3^{rd} ed., Cold Spring Harbor Laboratory Press). As will be understood, the term “recombinant” when referring to a nucleic acid molecule or construct means that heterologous nucleic acid sequences have been recombined, such that reference to a recombinant nucleic acid molecule refers to a molecule that is comprised of nucleic acid sequences that are joined together or produced by means of molecular biological techniques.

In various embodiments, GFAP promoter allelic variants and derivatives may be substantially homologous in that they hybridize to all or part of the hGFAP promoter under moderate or stringent conditions. Hybridization to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (see Ausubel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y.). Generally, stringent conditions are selected to be about 5° C. lower than the thermal melting point for the specific sequence at a defined ionic strength and pH. Stringent hybridization may, for example, be conducted in 5×SSC and 50% formamide at 42° C. and washed in a wash buffer consisting of 0.1×SSC at 65° C. Washes for stringent hybridization may, for example, be of at least 15 minutes, 30 minutes, 45 minutes, 60 minutes, 75 minutes, 90 minutes, 105 minutes or 120 minutes or longer.

The degree of homology between sequences may also be expressed as a percentage of identity when the sequences are optimally aligned, meaning the occurrence of exact matches between the sequences. Optimal alignment of sequences for comparisons of identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence alignment may also be carried out using the BLAST algorithm, described in Altschul et al., 1990, J. Mol. Biol. 215:403-10 (using the published default settings). Software for performing BLAST analysis may be available through the National Center for Biotechnology Information (through the internet at http://www.ncbi.nlm.nih.gov/). In various embodiments, the variants and derivatives may be at least 50%, at least 80%, at least 90% or at least 95%, or at least 99% identical as determined using such algorithms. In various embodiments, the transgene may encode a marker molecule that may be useful for the identification of HSCs in vitro and/or in vivo. In various embodiments, the marker molecule is a marker protein.

A marker molecule is any molecule whose expression may be determined. The marker molecule may be a RNA, whose expression may be determined by known method based on nucleic acid hybridization, such as for example, RT-PCR. Generally, however, the marker molecule is a marker protein. As used herein, “marker protein” refers to a protein whose expression and/or subcellular localization may be readily determined, such as, for example, green fluorescent protein (GFP). DNA vectors encoding other fluorescent proteins, for example, blue, cyan, green, and yellow-green and red are commercially available (Clonetech). Other marker proteins would be known to a person skilled in the art. In different embodiments, the marker protein may be an enzyme whose expression may be readily determined by providing a specific substrate and detecting the products of enzymatic turnover. Examples of enzymatic marker proteins include, for example, β-galactosidase and luciferase. Other enzymatic marker proteins would be known to a person skilled in the art. In other embodiments, the marker protein may be any protein whose expression may be detected immunologically, for example, by providing a labeled antibody that specifically recognizes and binds the marker protein, or a fragment thereof. The antibody may be a polyclonal antibody or a monoclonal antibody and may be directly or indirectly labeled according to methods known in the art, such as, for example, labeling with a fluorescent dye and detecting expression of the marker protein by fluorescence microscopy. Other immunological-based detection methods, including, for example, immunogold staining, radiolabelling, and colourometric enzymatic precipitation would be known to a person skilled in the art. In specific embodiments, the marker protein is β-galactosidase.

In other embodiments, the transgene operably coupled to the GFAP promoter encodes a therapeutic product. As used herein, “therapeutic product” includes any expression product having clinical usefulness, such as a RNA or protein that is involved in disease prevention, treatment, or a RNA or protein that has a cell regulatory effect that is involved in disease prevention or treatment. The therapeutic product may be a polypeptide, including a protein or a peptide, a ribozyme, a siRNA, an antisense RNA or a microRNA.

The therapeutic product may have clinical usefulness in diseases or disorders caused or associated with hepatic fibrosis, including, for example, cirrhosis, hepatitis C infection, hepatitis B infection, steatohepatitis associated with alcohol or obesity, hemochromatosis, Wilson's disorder, primary biliary cirrhosis (PBC) and non-alcoholic steatohepatitis (NASH).

In various embodiments, the therapeutic product is an anti-fibrotic polypeptide. Various anti-fibrotic polypeptides would be known to a person skilled in the art. Without being limited to any particular theory, the anti-fibrotic peptide may reduce or inhibit fibrosis by: (a) reducing inflammation to avoid stimulating HSC activation, such as, for example TNF-α antagonists and interleukin-10 (IL-10); (b) directly down-regulating HSC activation, such as for example, γ-interferon and hepatocyte growth factor (HGF); (c) neutralize proliferative, fibrogenic, contractile or pro-inflammatory responses of stellate cells, such as, for example, antagonists to PDGF, FGF or TGFα, including soluble cognate receptor fragments; (d) induce HSC apoptosis such as, for example, Bcl-xL or Fas; or (e) induce ECM degradation, such as, for example, matrix metalloprotease-8 (MMP-8).

In a specific embodiment, the therapeutic product is IL-10. Other therapeutic products would be known to a person skilled in the art and include, for example, the products of the Smad 7 gene, and the product of dominant negative alleles of Smad 3, Smad 4 and TGFR. Dominant negative forms of the Smad proteins are known and may be created by serine to alanine substitutions in the phosphorylation site, for example, by replacing SSXS sites to AAXA, where X represents any amino acid. Dominant negative forms of TGFR would also be known to a person skilled in the art. Other therapeutic products include, for example, dominant negative alleles of the platelet-derived growth factor receptor (PDGFR) and Diptheria toxin.

As would be understood by a person skilled in the art, a “dominant negative allele” is an allele whose expression inhibits or reduces the biological effect of the expression product of a wild-type allele. Without being limited to any particular theory, the product of a dominant negative allele may form an inactive heteromeric complex with the product of the wild-type allele.

In other embodiments, the therapeutic product is a siRNA. siRNAs are generally double stranded 19 to 22 nucleotide sequences that can effect post-transcriptional silencing of cognate mRNAs, allowing for selective suppression of gene expression. Generally, and without being limited to any specific theory, the sequence of the siRNA therapeutic product will be complementary to a portion of the mRNA of the gene sought to be silenced. For example, the siRNA may be designed to hybridize with mRNA encoding TGF-β1. HSC cells are the most important source of TGF-β1 in liver fibrosis and inhibiting TGF-β may inhibit matrix production and accelerate its degradation (Freidman (2003), J. Hepatol. 38 Suppl 1:S38). In other embodiments, the siRNA may be designed to hybridize against α1(I) collagen mRNA. Increased α1(I) collagen expression in HSCs has been shown to be mediated primarily through a post-transcriptional mechanism, with the half life of α1(I) collagen mRNA increasing from 1.5 hours in quiescent cells to greater than 24 hours in activated HSCs. In yet other embodiments, the siRNA may be designed to hybridize against nucleic acids encoding platelet-derived growth factor (PDGF) or extracellular matrix molecules, such as, for example, fibronectin, laminin and integrin.

Guidelines for designing siRNAs would be known to the person skilled in the art, or siRNA designed to hybridize to a specific target may be obtained commercially (Ambion, Qiagen). For example, siRNAs with a 3′ UU dinucleotide overhang are often more effective in inducing RNA interference (RNAi). Other considerations in designing siRNAs would be known to a person skilled in the art.

In another embodiment, isolated HSCs comprising a transgene operably coupled to GFAP promoter as described herein are also contemplated. The isolated transgenic HSC according to different embodiments may be used to study fibrogenesis, hepatic fibrosis or for developing anti-fibrosis gene therapies. In some embodiments, the transgenic HSC cell is a transgenic rat HSC-T6 cell and in specific embodiments the transgenic HSC cell is T6/lacZ/C1.

As used herein, “isolated” refers to transgenic HSCs in in vitro culture, in the presence or absence of other cell types.

In other embodiments, a transgenic HSC according to various aspects of the invention, other than an in vivo mouse transgenic HSC, are contemplated.

There is also presently provided a method for in vitro and/or in vivo identification of fibrogenesis modulating agents, using the GFAP promoter as described herein. Fibrogenesis modulating agents include agents that increase, upregulate or activate fibrogenesis, as well as agents that decrease, downregulate or inhibit fibrogenesis. Thus, fibrogenesis modulating agents include pro-fibrotic agents as well as anti-fibrotic agents.

In one embodiment the method allows for identification of anti-fibrotic agents. Thus, the method comprises detecting the expression levels of a gene that is operably coupled to a GFAP promoter in an HSC, in the presence and the absence of a putative anti-fibrotic agent.

The two expression levels are then compared and anti-fibrotic agents are identified where the expression of the gene is reduced or abolished in the presence of the agent. Gene expression levels may be detected by known methods, such as, for example, by the methods described herein for detecting expression of a gene, including a transgene.

In the present method, the gene operably coupled to the GFAP promoter may be an endogenous gene encoding the naturally GFAP protein. For example, the expression may be performed using a vector containing a region that includes the GFAP promoter and coding region. The expression may also be performed in an HSC, using the endogenous GFAP promoter and coding region. The levels of expression of the endogenous GFAP gene may be detected using methods known in the art, including by detecting levels of mRNA, for example by Northern blot, quantitative RT-PCR, RNA run-on assays, gene chip analysis or by any other methods known in the art for detecting mRNA levels in a cell or in a cell-free expression system.

Alternatively, the gene operably coupled to the GFAP promoter may be a transgene as described herein. For example, the expression may be performed using a vector containing the GFAP promoter operably coupled to a reporter gene that encodes a detectable product, including a detectable mRNA or a detectable protein. Such an expression vector may be used in a cell free system or in a transgenic HSC that has been transfected with the expression vector. mRNA levels may be detected as described herein, or a protein product may be detected as described herein, including by immunoassay techniques such as immunoblots or ELISAs, by radioassays, by chemiluminescence assays, by optical methods, by calorimetric methods, by fluorogenic methods, or by enzymatic methods, or by other methods known in the art for detecting levels of transgenic products.

In a particular embodiment, the GFAP promoter is operably coupled to the endogenous GFAP coding region, resulting in production of the GFAP mRNA transcript and ultimately the GFAP protein. In another particular embodiment, the GFAP promoter is operably coupled to the lacZ reporter transgene, resulting in production of the α-galactosidase mRNA transcript and ultimately in the β-galactosidase protein. Detection of β-galactosidase is well known in the art, and kits for such detection are commercially available, including chemiluminescent assay kits (e.g. available from Roche, Mannheim, Germany).

The expression may be conducted in an in vitro expression system. Cell-free expression systems are known in the art and are commercially available, and typically include all of the required enzymes and substrates to produce a transcript and/or a protein, including an RNA polymerase that is capable of transcribing the GFAP promoter, ribonucleotides, ribozomes, amino acids, tRNAs. Conducting the present assay in a cell free system would allow for screening and identification of anti-fibrotic agents that directly interact with the GFAP promoter.

The compounds that are to be screened as putative anti-fibrotic agents may be added directly to the in vitro system, and the expression levels of the gene operably coupled to the GFAP promoter are measured in the presence and absence of the putative anti-fibrotic agent. As stated above, an anti-fibrotic agent will result in measurable decreased expression from the GFAP promoter as compared to expression in the absence of the anti-fibrotic agent.

However, it may be desirable to identify anti-fibrotic agents that act indirectly on the GFAP expression levels, for example by acting further upstream in a pathway that activates GFAP expression. Thus, the expression may be performed in a cultured cell, wherein the cultured cell is any cell containing the GFAP promoter operably coupled to a gene including a transgene, and particularly may be an HSC. As stated above, the GFAP promoter may be the endogenous GFAP promoter operably coupled to the endogenous GFAP coding region, or it may be a construct comprising the GFAP promoter operably coupled to a transgene in a transgenic HSC.

In the method performed in a cell, the putative anti-fibrotic agent may be contacted with the cell, and expression levels of the gene operably coupled to the GFAP promoter are then detected. As with the cell-free method, expression levels in the presence and in the absence of the putative anti-fibrotic agent are compared, with an anti-fibrotic agent providing measurably less expression than in the absence of the anti-fibrotic agent.

In a particular embodiment, the cells used are the HSC-T6 cell line (Vogel et al. (2000), J Lipid Res. 41(6):882) and the GFAP promoter is the endogenous promoter operably coupled to the endogenous GFAP coding sequence. In another particular embodiment, the HSC-T6 cells are transfected with a GFAP-lacZ expression construct, for example as in the T6/GFAP-lacZ cell line (Maubach et al., (2006) World J Gastroenterol. 12(5):723-30).

The method may be performed using known pro-fibrotic agents to elevate the levels of expression from the GFAP promoter. The known pro-fibrotic agent may be, for example, TGF-β1, PDGF-BB or lipopolysaccharide. The known pro-fibrotic agent may be administered to a cell in which the expression is to be performed, either prior to treatment with the putative anti-fibrotic agent, or concurrently with the putative pro-fibrotic agent.

For example, cells may be pre-treated with a pro-fibrotic agent for a given period sufficient to result in elevated expression from the GFAP promoter, for example from about 1 to about 3 days. The culture medium may then be replaced with fresh medium not containing any pro-fibrotic agent prior to treatment with the putative anti-fibrotic agent. Alternatively, the pro-fibrotic agent and the putative anti-fibrotic agent may be administered concurrently, for example by addition to the culture medium for a period from about 1 to about 3 days.

The putative anti-fibrotic agent may be administered to the cells for example by addition to the culture medium, for a given time period, for example from about 2 hours, to about 3 days.

The present method may also be performed in a cell in an in vivo context, for example in an animal model including a transgenic animal model, by administering the putative anti-fibrotic agent to the animal and comparing expression levels from the GFAP promoter in HSCs in the animal given the putative anti-fibrotic agent with expression levels from the GFAP promoter in animals not given the putative anti-fibrotic agent.

Administration of or exposure to the putative anti-fibrotic agent may be done in comparison with administration of a known anti-fibrotic agent. The known anti-fibrotic agent may be, for example, epigallocatechin gallate (EGCG), genistein or N-acetylcysteine.

The known anti-fibrotic agent may be used to develop a standard curve for measuring expression levels or reduction of expression levels in response to a given concentration of a known anti-fibrotic agent, as will be appreciated by a skilled person. For example, the known anti-fibrotic agent may be added to the culture medium at a final concentration of about 0.1 μM or greater, of about 100 μM or less, or from about 0.1 μM to about 100 μM. For example, a standard curve may include various concentrations of known anti-fibrotic agent such as 0.1 μM, 0.2 μM, 0.5 μM, 1 μM, 5 μM, 10 μM, 25 μM, 50 μM and 100 μM, or such as 0.01325 μM, 1.25 μM, 5 μM, 10 μM, 20 μM and 40 μM.

The concentration of putative anti-fibrotic agent to use can then be determined in relation to such a standard curve, adjusting the concentration or amount used so as to fall within the limits of the standard curve. A person skilled in the art could routinely determine the concentration of the putative anti-fibrotic agent to be used in the screen according to the in vitro or in vivo screening method. The appropriate amount of the putative anti-fibrotic agent employed in the screen will depend, among other things, on the nature of the anti-fibrotic agent. A person skilled in the art would know to determine the appropriate concentration range, for example, by screening over two or more orders of magnitude and would appreciate that more potent anti-fibrotic agents would decrease expression levels of the transgene at a lower concentration compared to a less potent anti-fibrotic agent.

The present method for identifying putative anti-fibrotic agents may be used to determine the effect of concurrent treatment with two or more known or putative anti-fibrotic agents. The method may be performed as generally described herein, with the two or more agents being administered to the expression system either sequentially or concurrently to determine whether treatment with two or more anti-fibrotic agents has a synergistic effect that is greater than the additive effects of the two or more agents when administered independently to the expression system.

The above-described method may be adapted for high-throughput screening of a large number of putative anti-fibrotic agents. For example, cells may be cultured in large numbers of individual batches, for example in 96-well culture plates. In this way, the method may be adapted for multiple simultaneous screening methods to be performed, using known automated techniques to conduct assays in parallel.

Any anti-fibrotic agents identified in the above-described methods may be used in the treatment of a disease or disorder in which hepatic fibrosis occurs, including hepatitis caused by: hepatitis viruses (e.g., HVB), drug toxicity (e.g., acetaminophen), liver toxins (e.g., aflatoxin B₁), alcoholic consumption, hepatic portal hypertension, bile duct obstruction, fatty liver (non-alcoholic steatohepatitis) or liver autoimmune disorders, and cirrhosis, portal hypertension liver cancer, hepatitis C infection, hepatitis B infection, PBC, NASH, hemochromatosis, Wilson's disorder or steatohepatitis associated with alcohol and obesity.

In another embodiment the method allows for identification of pro-fibrotic agents. Thus, the method comprises detecting the expression levels of a gene that is operably coupled to a GFAP promoter in an HSC, in the presence and the absence of a putative pro-fibrotic agent. The two expression levels are then compared and pro-fibrotic agents are identified where the expression of the gene is increased in the presence of the agent. Gene expression levels may be detected by as described herein.

As above, identification of pro-fibrotic agents may be performed in a cell-free system or in a cell, including in an isolated cell in culture or in a cell in an in vivo animal model.

Generally, identification of pro-fibrotic agents may be performed as described herein for screening for anti-fibrotic agents. A skilled person will understand that when comparing results with those obtained for a known agent, the known agent will be a known pro-fibrotic agent, for example transforming growth factor-β1 (TGF-β1), platelet derived growth factor-BB (PDGF-BB) or lipopolysaccharide (LPS).

In another embodiment, there is provided a method of diagnosing the presence of hepatic fibrosis in a subject or determining the prognosis of a subject having or being likely to develop hepatic fibrosis.

The subject may be any subject who currently has a disease or disorder caused by or characterized by the presence of hepatic fibrosis, who is likely to develop such a disease or disorder or in whom such a disease or disorder is desired to be prevented, including a human subject.

In one embodiment, HSCs obtained from the patient are cultured. Levels of expression of the endogenous hGFAP mRNA or endogenous hGFAP protein are then detected as is known in the art and as described herein, including using immunoassay methods to detect the GFAP protein, including in situ immuno-staining methods.

Alternatively, the cells from the patient and the cells not associated with hepatic fibrosis may be transfected with a vector comprising the GFAP promoter operably coupled to a transgene, as described herein.

The levels of expression from the GFAP promoter may be compared to levels of expression from the GFAP promoter in a cell that is not associated with hepatic fibrosis. Such a cell that is not associated with hepatic fibrosis may be from a healthy individual or from a cell line that has GFAP expression levels that are not increased due to activation of the HSC. The levels of expression from the GFAP promoter may be higher in cells from a subject that has hepatic fibrosis than in cells not associated with hepatic fibrosis, and may be higher be in cells from a subject prone to or predisposed to developing hepatic fibrosis.

The GFAP expression levels may be compared in the presence and absence of a known fibrogenesis modulating agent. The fibrogenesis modulating agent may be an anti-fibrotic agent or a pro-fibrotic agent as described herein. As will be understood, in cells from a subject who has, is predisposed to, or is likely to develop a disease or disorder caused by or characterized by the presence of hepatic fibrosis, contacting the cell with a pro-fibrotic agent tends to result in increased expression from the GFAP promoter. Similarly, contacting the cells with an anti-fibrotic agent tends to result in decreased expression from the GFAP promoter.

In another embodiment, there is provided a method for treating a disorder characterized or caused by hepatic fibrosis including, for example, cirrhosis, portal hypertension liver cancer, hepatitis C infection, hepatitis B infection, PBC, NASH, hemochromatosis, Wilson's disorder or steatohepatitis associated with alcohol and obesity, hepatitis including hepatitis caused by hepatitis caused by: hepatitis viruses (e.g., HVB), drug toxicity (e.g., acetaminophen), liver toxins (e.g., aflatoxin B₁), alcoholic consumption, hepatic portal hypertension, bile duct obstruction, fatty liver (non-alcoholic steatohepatitis) or liver autoimmune disorders.

The method includes administering to a subject a nucleic acid encoding a therapeutic product operably coupled to a HSC-specific hGFAP promoter, according to various aspects of the invention. In specific embodiments, the hGFAP promoter is the 2.2 kb promoter fragment of SEQ ID NO:1. In certain embodiments, the therapeutic product is an anti-fibrotic molecule, and in specific embodiments is an anti-fibrotic polypeptide.

The subject is any subject in need of such treatment, including a mammal, and particularly a human subject.

To deliver the nucleic acid molecule specifically to HSCs, the nucleic acid may be delivered by methods known in the art, for example, by the hydrodynamic delivery of therapeutic genes via the afferent and efferent vessels of the liver, such as for example, the portal vein, the hepatic vein, or the bile duct.

Methods for introducing the nucleic acid molecule into mammalian cells in vivo are known, and may be used to administer the nucleic acid vector of the invention to a subject. A nucleic acid may be delivered into a HSC by direct injection of DNA, receptor mediated DNA uptake, viral-mediated transfection or non-viral lipid based transfection. The nucleic acid vector may be administered by microparticle bombardments, for example, using a commercially available “gene gun” (BioRad).

The nucleic acid molecule is administered in such amounts to achieve the desired results, for example, the expression of the therapeutic transgene in HSCs. For example, the nucleic acid may be delivered in such amounts to express sufficient amounts of the therapeutic product which functions to alleviate, mitigate, ameliorate, inhibit, stabilize, improve, prevent, including slow the progression of the disorder, the frequency of treatment and the type of concurrent treatment, if any.

To aid in administration, the nucleic acid molecule may be formulated as an ingredient in a pharmaceutical composition. The compositions may routinely contain pharmaceutically acceptable concentrations of salt, buffering agents, preservatives and various compatible carriers or diluents. For all forms of delivery, the nucleic acid molecule may be formulated in a physiological salt solution.

The proportion and identity of the pharmaceutically acceptable diluent is determined by chosen route of administration, compatibility with a nucleic acid molecule, compatibility with a live virus when appropriate, and standard pharmaceutical practice. Generally, the pharmaceutical composition will be formulated with components that will not significantly impair the biological properties of the nucleic acid. Suitable vehicles and diluents are described, for example, in Remington's Pharmaceutical Sciences (Remington's Pharmaceutical Sciences, Mack Publishing Company, Easton, Pa., USA 1985).

Solutions of the nucleic acid molecule may be prepared in a physiologically suitable buffer. Under ordinary conditions of storage and use, these preparations contain a preservative to prevent the growth of microorganisms, but that will not inactivate or degrade the nucleic acid molecule. A person skilled in the art would know how to prepare suitable formulations. Conventional procedures and ingredients for the selection and preparation of suitable formulations are described, for example, in Remington's Pharmaceutical Sciences and in The United States Pharmacopeia: The National Formulary (USP 24 NF19) published in 1999.

The forms of the pharmaceutical composition suitable for injectable use include sterile aqueous solutions or dispersion and sterile powders for the extemporaneous preparation of sterile injectable solutions or dispersions, wherein the term sterile does not extend to any live virus that may comprise the nucleic acid molecule that is to be administered. In all cases the form must be sterile and must be fluid to the extent that easy syringability exists.

In another embodiment, there is provided a method for treating a disorder characterized or caused by liver fibrosis, the method comprising administering to a subject in need thereof, a transgenic HSC according to various aspects of the invention, wherein the transgenic HSC comprises a transgene encoding a therapeutic product.

The subject is any subject suffering from a disorder characterized or caused by liver fibrosis, including, for example, cirrhosis, portal hypertension, liver cancer, hepatitis C infection, hepatitis B infection, PBC, NASH, hemochromatosis, Wilson's disorder or steatohepatitis associated with alcohol and obesity and who is in need of such treatment. The patient may be any animal, including a mammal, particularly a human.

An effective amount of transgenic HSCs may be administered to subject, using methods known in the art, including by surgical implantation or by injection in or near the subject's liver. The term “effective amount” as used herein means an amount effective, at dosages and for periods of time necessary to achieve the desired result, for example, to treat the specific disorder. The number of total transgenic HSCs to be administered will vary, depending on, among other things, the disorder or disease to be treated, the mode of administration, the age and health of the patient and the expression levels of the therapeutic product.

Kits and commercial packages containing the various nucleic acid molecule constructs described herein, including an expression vector containing a coding sequence encoding a therapeutic product operably coupled to a promoter comprising a HSC-specific GFAP promoter, or kits and commercial packages containing a pharmaceutical composition as described herein, are contemplated. Such a kit or commercial package will also contain instructions regarding use of the included nucleic acid molecule or pharmaceutical composition, for example, use to diagnose or treat a hepatic-fibrosis related disorder, for example or for expressing an expression product in a HSC, including in accordance with the methods described herein.

The invention is further exemplified by the following non-limiting examples.

### EXAMPLES

### Example 1

Materials and Methods

Construction of the 2.2 kb hGFAP-lacZ Transgene

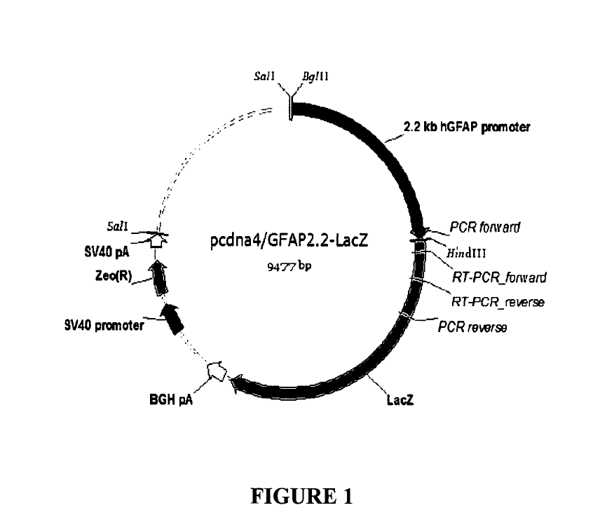

The plasmid vector pcDNA4/TO/LacZ (Invitrogen, CA, USA) was used as a cloning backbone, providing the E. coli β-galactosidase coding sequence and the Zeocin resistance gene. The 2.2 kb human GFAP promoter used in this experiment was originally mapped for its astrocyte-specific expression both in vitro (Besnard et al. (1991), J Biol Chem. 266(28):18877) and in vivo (Brenner et al. (1994), J Neurosci. 14: 1030). The CMVTetO2 promoter in the pcDNA4/TO/lacZ vector was replaced with the 2.2 kb GFAP promoter, which was excised from another transgene GFAP-GFP-S65T (Zhuo et al. (1997), Developmental Biology 187:36) by BglII/HindIII digest. The promoter/coding region junction sequences were verified by DNA sequencing. The resultant 9.5 kb plasmid (as depicted in FIG. 1) was designated as pcDNA4/GFAP2.2-LacZ in our vector depository.

Cell Lines and Culture Conditions

The rat HSC-T6 cell line (Vogel et al. (2000), J Lipid Res. 41(6):882) was a generous gift from Dr. Scott Friedman of the Mount Sinai School of Medicine in New York through Dr. Alex Hui (Chinese University of Hong Kong). The HepG2, C3A (a clonal derivative of the HepG2), HeLa and NIH/3T3 were from ATCC (American Type Culture Collection, VA, USA). The C6 was from JCRB (Japanese Collection of Research Bioresources, Osaka, Japan). All the cell culture media and reagents were purchased from Invitrogen (Carlsbad, Calif., USA), unless specified otherwise. All cell lines were routinely cultured in DMEM (Dulbecco's Modified Eagle Medium), supplemented with 10% FBS and 100 units penicillin/100 microgram streptomycin per ml (full DMEM) at 37° C. in a humidified atmosphere of 5% CO₂. The cells were routinely split twice a week in a 1:3 ratio by trypsinization (0.05% trypsin/0.53 mM EDTA).

Cell Transfection and Stable Selection

The T6, C6 and HeLa cells were stably transfected with 0.5-1.0 μg of the linearised plasmid vector using the Lipofectamine-2000 kit, according to instructions provided by the manufacturer (Invitrogen, CA, USA). Twenty-four hours after transfection, the medium was changed and supplemented with 250-500 μg/ml Zeocin™ for selection for at least six weeks before further assays as described below are performed. The final concentration for maintaining the stable transfected cells was 500 μg/ml Zeocin™. Alternatively, HepG2, C3A and NIH/3T3 were transiently transfected with the circular plasmid using the FuGene 6 kit (Roche Diagnostics).

Genomic Detection of the Transgene

In order to confirm the transgene integrity and integration into the genome in stable transfectants, genomic DNA was isolated from cell clones stable for at least two months under 500 μg/ml Zeocin selection, using the NucleoSpin blood kit (Macherey & Nagel, Düren, Germany). A polymerase chain reaction (PCR) strategy was used to verify the structural integrity of the inserted construct(s), with a forward primer 5′-ACTCCTTCATAAAGCCCTCG-3′ [SEQ ID NO: 2] (complementary to the GFAP promoter), and a reverse primer 5′-AACTCGCCGCACATCTGAACTTCAGC-3′ [SEQ ID NO: 3] (complementary to the lacZ coding sequence). The Platinum PCR SuperMix High fidelity (Invitrogen, CA, USA) was used to carry out the DNA amplification on a thermal cycler (MJ Research, FL, USA). The PCR product was analyzed on a 1% agarose gel in the RunOne electrophoresis system (EmbiTec, CA, USA) in 0.5×TAE buffer. The finished gel was documented with the AlphaDigiDoc photo system (Alpha Innotech, CA, USA). The expected PCR product was 944 bp in size.

RT-PCR GFAP-lacZ Transcript

Total cellular RNA was extracted from cells grown in 6-well plates by using the NucleoSpin RNAII kit (Macherey-Nagel, Düren, Germany) following the manufacturer's instructions. The RNA concentration was determined on a ND-100 spectrophotometer (Nanodrop Technologies, DE, USA). Fifty nanograms of total RNA was used to perform a one-step RT-PCR (Qiagen, Hilden, Germany) according to the user's manual. The primers used were: forward 5′-TCAGCTTGGAGTTGATCCCGTCG-3′ [SEQ ID NO: 4], reverse 5′-AACAAACGGCGGATTGACCGTAATGG-3′ [SEQ ID NO: 5]. The reaction conditions were 50° C. 30 min (cDNA synthesis), 95° C. 15 min (denaturation), 94° C. 10 sec, 55° C. 10 sec, 72° C. 19 sec for 35 cycles (PCR). The product size (337 bp) was verified in a 3% agarose gel. The β-actin (332 bp) was used as a reference.

X-Gal Staining of β-Galactosidase Activity in Fixed Cells

A lacZ reporter assay kit for cell staining (InvivoGen, CA, USA) was used to detect the reporter gene activity in both stable and transient transfectants. Development of the blue end product was monitored at different time intervals (15, 30, 60, 90, and 120 minutes). The staining results were documented with a digital camera (DP12) attached to an Olympus inverted bright field microscope (IX51).

Quantitative Solution Assay of β-Galactosidase Activity in Cell Extracts

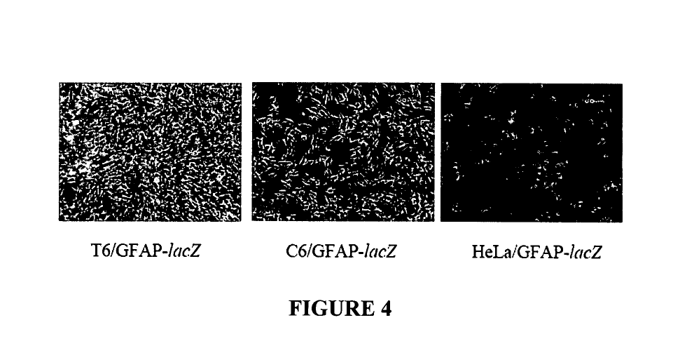

To quantify the β-galactosidase activity in cell extracts reported in FIGS. 4 to 10, an enzyme assay kit (E2000, Promega, Wis., USA) was used to measure the specific activity of P-galactosidase, according to the manufacturer's instructions in a 96-well format. Briefly, the cells were washed twice with 1×PBS buffer (pH7.4), lysed for 15 minutes at room temperature with the reporter lysis buffer, and harvested using a cell scraper. The total cell extracts were appropriately diluted (5-10 fold), and assayed for the enzyme activity. The specific β-galactosidase activity was expressed in milliunit per milliliter (mU/ml) using a standard curve established from the β-galactosidase standard (provided with the kit). The protein concentration was measured with a BCA protein assay kit (Pierce, Ill., USA), and used to further convert the specific β-galactosidase activity from mU/ml to mU/μg total cellular protein. Six independent experiments were performed for each time and concentration data point.

Treatment of Cells with TGF-β1, PDGF-BB and LPS

Cells were seeded into 12-well plates in full DMEM and 500 μg/ml Zeocin. Twelve hours prior to the cytokine treatment, medium was changed so that the cells were allowed to grow in low serum (0.5% FBS) DMEM. The cells at a confluence of 70-80% were incubated with various concentrations (0, 0.1, 1 and 10 ng/ml) of recombinant human TGF-β1, PDGF-BB (BioVision, CA, USA) and 0, 0.1, 1 and 10 μg/ml lipopolysaccharide (LPS E. coli, Sigma) respectively for 0, 2, 8, 16, 24, 48 and 72 hours before they were harvested for β-galactosidase activity assay and real-time RT-PCR GFAP assay.

Real-Time RT-PCR for Endogenous Rat GFAP mRNA

Total RNA was reverse transcribed to cDNA using Taqman's reverse transcription reagent (Cat. #N₈O₈-0234A total of 400 ng of RNA in 7.7 μl nuclease-free water was added to 2 μl 10× reverse transcriptase buffer, 4.4 μl 25 mM magnesium chloride, 4 μl deoxyNTP mixtures, 1 μl random hexamers, 0.4 μl RNase inhibitor and 0.5 μl reverse transcriptase (50 U/μl) in a final reaction volume of 20 μl. The reaction was performed for 10 min at 25° C. (annealing), 30 min at 48° C. (cDNA synthesis) and 5 min at 95° C. (enzyme denaturation).

Real-time quantitative PCR was carried out with an ABI 7500 Real Time PCR System (Applied Biosystems, CA, USA). One microliter of sample cDNA was used in each PCR reaction, with the housekeeping β-actin gene as a reference for normalization. The primers and probes for rat β-actin and GFAP were purchased from Taqman's assay-on-demand database. The PCR reaction was performed under a default profile consisting of 50° C. for 2 min (UNG activation), 95° C. for 10 min (enzyme denaturation) and 40 cycles of amplification (denaturation 15 seconds, annealing and extension 60 seconds).

Relative quantitation of the target mRNA was calculated using the comparative threshold cycle (C_{T}) methods as described in the User Bulletin #2 (ABI Prism 7700 Sequence Detection System). C_{T} indicates the fractional cycle number at which the amount of amplified target reaches a fixed threshold within the linear phase of gene amplification. ΔC_{T}, which reflects the difference between C_{T target} and C_{T β-actin}, is inversely correlated to the abundance of mRNA transcripts in the samples. ΔC_{T} for each sample was normalized against control experiment or calibrator and expressed as ΔΔC_{T}. Relative quantitation is given by 2^{-ΔΔCT} to express the up-regulation or down regulation of the target gene under the treatments compared to the control. Six independent experiments were performed for each data point and three ACT were measured for each experiment.

Statistical Analysis

All quantitative results were presented as mean ±SE. Experimental data were analyzed using two-tailed Student's t-test assuming unequal variances. A P-value≦0.05 was considered statistically significant.

Transgenic GFAP-GFP Mice and Immunohistochemistry

The generation and genotyping of the transgenic GFAP-GFP mice were done as previously described (Zhuo (1997), Developmental Biology 187:36). Mice were perfused with 0.1 M phosphate buffer saline (PBS, pH 7.4) and 4% paraformaldehyde. Liver was harvested and soaked in 30% sucrose at 4° C. overnight. Then the liver was embedded for cryosectioning using a cryostat (Leica Microsystems, Germany). For GFP and GFAP immunostaining, a mouse anti-GFP monoclonal antibody (Clontech, USA) and a rabbit anti-GFAP polyclonal antibody (DakoCytomation, Denmark; Z-0334) were used. The cryosections were washed for 5 min in 0.15 M 1×PBS followed by incubating for 4 hr at 4° C. in blocking solution of PBS containing 0.1% (v/v) Triton X-100 and 10% nonimmune goat serum. The cryosections were then washed in 0.15 M 1×PBS three times, each for 15 min. The liver sections were then incubated overnight with anti-GFP antibody (1:200) and anti-GFAP antibody (1:200) in 1×PBS containing 0.01% (v/v) Triton X-100 and 1% nonimmune goat serum at 4° C. After 3×15 min rinse in 1×PBS, the sections were incubated with a goat anti-mouse IgG conjugated with FITC (Sigma Chemicals, USA) and a goat anti-rabbit IgG conjugated with Texas-red (Abcam, U.K.) at 1:100 dilution in PBS containing 0.01% (v/v) Triton X-100 and 1% nonimmune goat serum for 2 hr at room temperature. After 3×15 min rinse in 1×PBS, the sections were coverslipped in 10 μl of the fluorescence medium and photographed with a confocal laser scanning microscope (Olympus, Japan).

Results

Transient Transfection of Non-GFAP Expressing Cell Lines with GFAP-lacZ

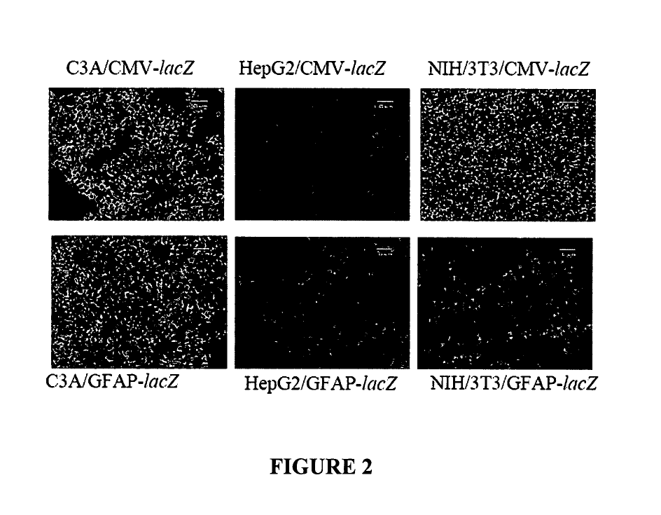

To investigate if there is any possible aberrant expression of the 2.2 kb hGFAP-lacZ transgene in several commonly used non-GFAP expressing cell types, we transiently transfected three cell lines (HepG2, C3A and NIH/3T3) with a circular form of the transgene, using a CMV-lacZ plasmid (Invitrogen) as a positive control. After the transfected cells were grown in full DMEM medium free of selection agent for two days, all three cell lines were examined for the expression of the β-galactosidase by X-gal staining method. Approximately 10-30% of the cells in each of the three lines transfected with CMV-lacZ showed blue staining (indicative of lacZ expression) after two hours of X-gal staining. In contrast, not a single cell in any of three lines transfected with the GFAP-lacZ showed any blue staining. This indicates a total lack of lacZ expression driven by the GFAP promoter in these cell lines. Representative images from two independent experiments for all three cell lines were shown in FIG. 2. After 24 hours of X-gal staining, a few cells from the HepG2 line displayed blue color in both the transfected and nontransfected groups alike (data not shown), indicative of endogenous galactosidase-like activity, which was unrelated to the transgene. No blue cells were observed in the transfected C3A and NIH/3T3.

Stable Cell Line Transfection, Selection and Cell Specificity of Transgene Expression

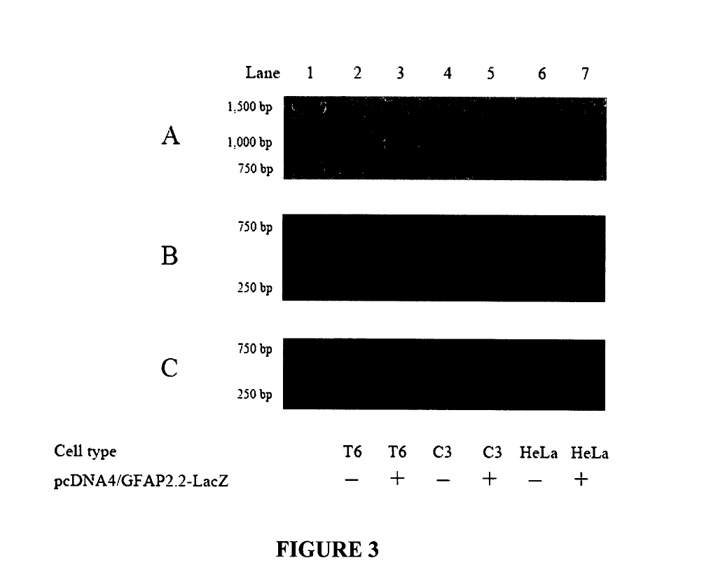

It was unknown whether a GFAP promoter can direct gene expression specifically in the hepatic stellate cell type. We decided to test this possibility by stably transfecting the rat hepatic stellate cell line T6 (Vogel et al. (2000), J Lipid Res. 41(6):882) with the GFAP-lacZ transgene, using the rat astrocyte C6 cell line and the human HeLa cell line as a positive and a negative control respectively. After selection in 250-500 mg/ml Zeocin for at least 6 weeks, several independent stable cell clones were obtained for each cell line. Subsequently, genomic DNA was isolated for analysis by PCR to check for the transgene integrity and successful integration into the host genome. PCR results confirmed the intact transgene integration in all cell lines, as evidenced by the presence of an anticipated product size of 944 bp (FIG. 3A). Furthermore, total RNA was isolated from the stable transfectants and RT-PCR was performed to verify for the lacZ transcript. The presence of a specific band with a predicted size of 337 bp indicated the lacZ transcription in the positive control C6 line, and more importantly in our target T6 line as well, but not in the negative control line HeLa (FIG. 3B). The β-actin sample is shown in FIG. 3C as a control for equal RNA loading. Next, one clone from each of the three cell lines was randomly chosen for X-gal staining for one hour. As shown in FIG. 4, the C6 and the T6 cells were positive and the HeLa was negative in blue staining. Therefore for the first time, a GFAP-based reporter gene was shown to specifically express in a hepatic stellate cell line. From this point onward, a T6 cell clone stably transfected with the 2.2 kb hGFAP-lacZ transgene (designated as T6/lacZ/C1 clone) was used for further experiments described below.

To our best knowledge, this is the first report that a GFAP promoter can direct transgene expression specifically in a hepatic stellate cell type, in a similar fashion as the endogenous GFAP does. Based on our transfection results of multiple cell types, the 2.2 kb hGFAP promoter was sufficient to confer HSC-specific, pro-fibrotic induceable expression in vitro.

X-Gal Staining of T6/lacZ/C1 Cells Treated with TGF-β1, PDGF-BB and LPS

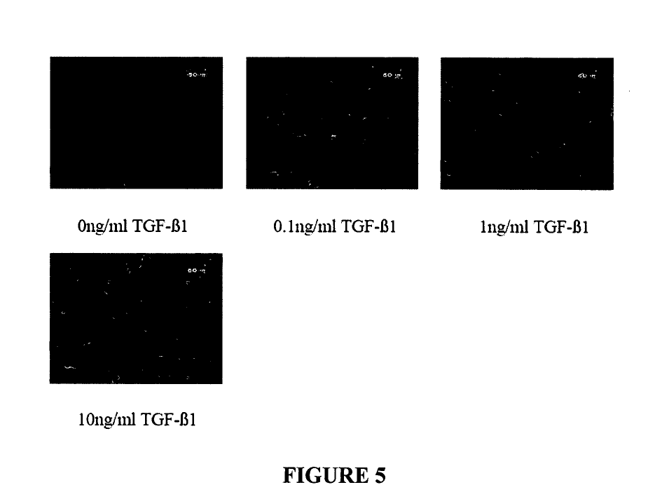

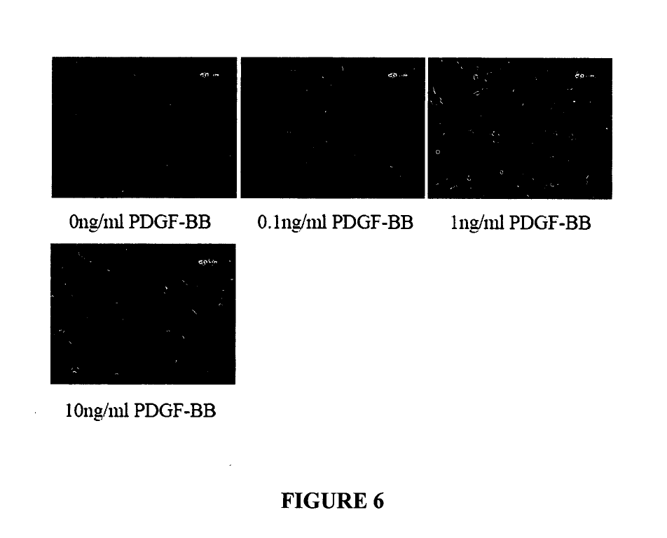

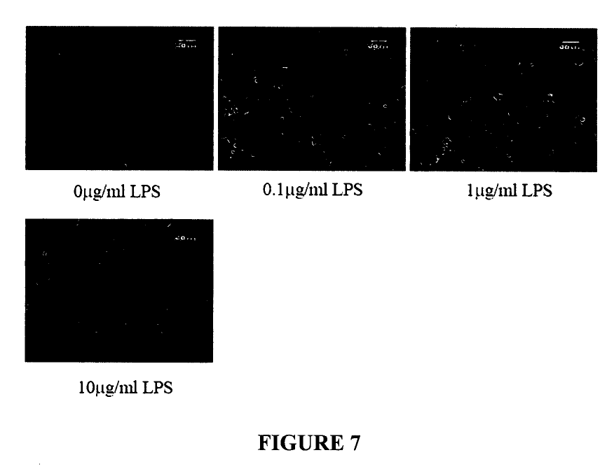

To determine possible induction of GFAP-lacZ expression by molecules with pro-fibrotic and proinflammatory properties in activated HSC the T6/lacZ/C1 cells were treated with TGF-β1, PDGF-BB and LPS respectively at various concentrations (0, 0.1, 1 and 10 ng/ml) in the full DMEM medium for 72 hours. The cells were then stained with X-gal for one hour and photographed. When compared to the untreated, cells treated with the pro-fibrotic TGF-β1 (FIG. 5) and the proinflammatory LPS (FIG. 7) displayed more intense blue staining starting from 0.1 to 10 ng/ml, while the pro-proliferative PDGF-BB (FIG. 6) showed less pronounced response. In order to obtain truly quantitative data, a solution assay was employed to measure possible up-regulation of β-galactosidase activity by TGF-β1 in experiments below.

Quantitation of β-Galactosidase Activity in T6/lacZ/C1 and C6/lacZ/C4 Without Cytokine Treatment

A β-galactosidase activity assay kit (Promega, Wis., USA) was used to measure transgene expression level in the T6/lacZ/C1 clone, using the nontransfected T6 as a negative basal control. When grown in the full DMEM medium to 70-80% confluence, the T6/lacZ/C1 cells yielded an average reading of approximately 0.5 mU/μg, about five times of that in normal T6 cells. The basal reading in the T6 cells was not influenced by the addition of cytokines or LPS (data not shown). For comparison purpose, the β-galactosidase activity in a randomly selected C6 clone containing the 2.2 kb hGFAP-lacZ transgene (designated as C6/lacZ/C4) was measured to have an activity of about 2 mU/μg, three times of that in the T6/lacZ/C1 cells. When incubated with TGF-β1 (0 to 10 ng/ml), the C6/lacZ/C4 cells yielded an induction dynamics of 130 to 200% of the untreated control. Under similar conditions, the T6/lacZ/C1 displayed analogous results (see data below). These data validated the use of the current assay method for quantifying transgene induction by cytokines.

Time- and Dose-Dependent β-Galactosidase Activity in Response to TGF-β1 Stimulation

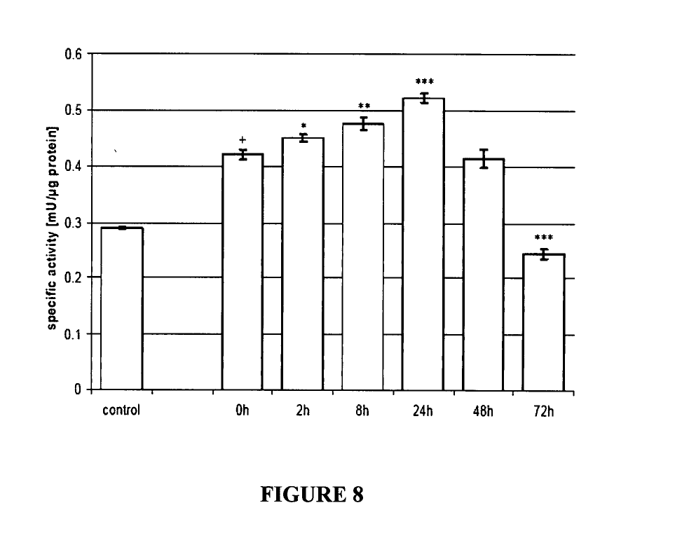

To quantify specific transgene response to TGF-β1, we first set up an experiment to assess possible effects that serum concentration and culture duration may have on the transgene expression. T6/lacZ/C1 cells were initially grown in DMEM with high serum (10% FBS), and then some of the cells were starved with a DMEM with low serum (0.5% FBS) for 12 hours prior to the addition of TGF-β1 (1 ng/ml). After incubation for various times (0, 2, 8, 24, 48 and 72 hours), cells were harvested for β-galactosidase activity assay. Surprisingly, the transgene expression significantly (P<0.001) increased from 0.29 mU/μg to 0.42 mU/μg (or a 24% increase) by simply lowering the serum concentration from 10% to 0.5% for 12 hours. After the TGF-β1 addition (to the medium with 0.5% FBS) at 0 hour, the transgene expression level was rapidly elevated at 2 and 8 hours, and eventually peaked at 24 hour, with a specific activity being 0.52 mU/μg. At 48 hours, the expression level dropped to the same level as at 0 hour. After 72 hours, the level slipped to 58% of the value at 0 hour (P<0.001). The assay results were depicted in FIG. 8.

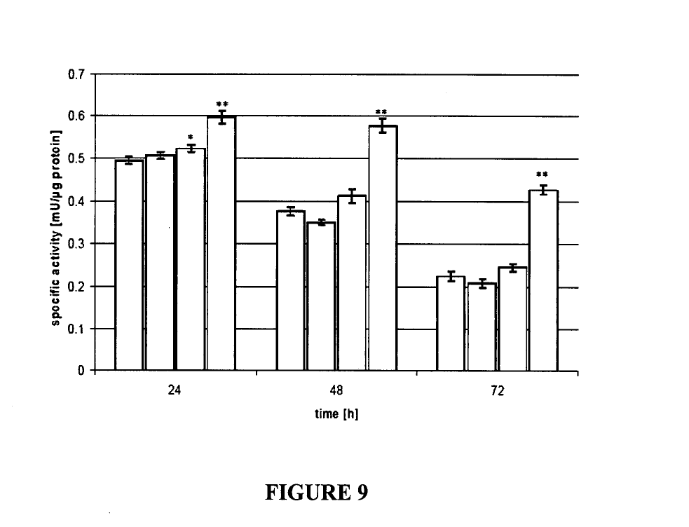

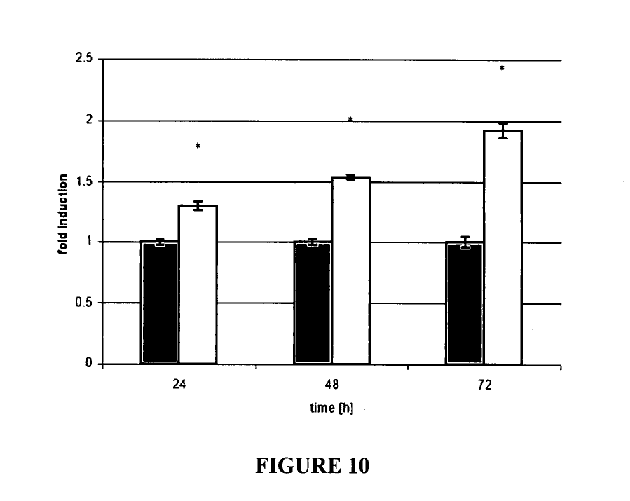

To further dissect the TGF-β1 contribution to the transgene induction, we treated T6/lacZ/C1 cells grown in low serum with various concentrations of TGF-β1 (0, 0.1, 1, 10 ng/ml) and assayed the enzymatic activity for three time points (24, 48 and 72 hours). The assay results were plotted in FIG. 9. Significant induction was observed for the 10 ng/ml TGF-β1 treatment at all time points (P<0.001). The only other (and less) induction was seen with 1 ng/ml TGF-β1 at 24 hours (P<0.05). When the non-treatment groups were normalized to 1 and the magnitude of induction were plotted for the 10 ng/ml treatment groups at all three time points (FIG. 10), a trend of increasing induction with time was apparent. The most robust induction (nearly two fold) was recorded at 72 hours, though the absolute enzymatic activity (0.43 mU/μg) was the lowest among the three time points.

Time-Dependent Induction of Endogenous GFAP Transcript by TGF-β1 as Measured by Real-Time RT-PCR

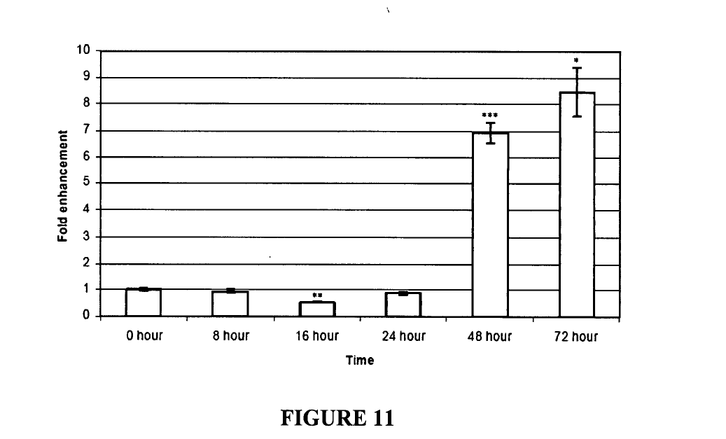

It was known that GFAP, along with desmin, SMAA, and vimentin, was expressed in the clonal T6 cells (Vogel et al. (2000), J Lipid Res. 41(6):882). Naturally, the endogenous GFAP should be expressed in the T6/lacZ/C1 cells as well. In order to investigate how the endogenous GFAP expression is regulated by TGF-β1 in the T6/lacZ/C1 cells during different time course, we quantified rat GFAP mRNA level using a real-time RT-PCR method. Cells were similarly grown and treated with TGF-β1 (1 ng/ml) for various times (0, 2, 8, 16, 24, 48 and 72 hours), as described for the enzymatic assay experiments above. Total RNA was isolated from different cells and quantified as described in Materials and Methods, and the results were depicted in FIG. 11. When compared to the basal level (normalized to 1) at 0 hour, the GFAP mRNA level remained steady within the first 24 hours, except a brief but significant suppression (0.5 fold) at 16 hour (P<0.005), then sharply increased to 7 and 8.5 fold of the basal level at 48 and 72 hours respectively.

The endogenous GFAP gene displayed a similar trend in response to TGF-β1 as the 2.2 kb GFAP-lacZ transgene did. However, two different responding features were noted between the native and the surrogate genes. First, the transgene induction was obvious even after 2 hours of incubation with TGF-β1, while the endogenous gene did not show any induction during the first 24 hours of TGF-β1 incubation. The difference in induction time line may be partially caused by the different assay detection sensitivity, presumably with the more sensitive enzymatic assay (with amplification) showing an earlier induction. Alternatively or in addition, unknown regulatory elements residing outside the 2.2 kb fragment may contribute to a more complex regulation and the observed brief decrease and delayed induction of the endogenous GFAP (FIG. 11).

As described herein, the 2.2 kb hGFAP promoter is capable of expressing the lacZ reporter specifically in the rat hepatic stellate cell line T6 and responding to TGF-β1 stimulation during cell activation by up-regulation of the transgene expression. This suggests that all the cis-acting and trans-acting components and pathways needed for expression and induction of the transgene are preserved within the 2.2 kb hGFAP promoter and conserved in cultured HSCs.

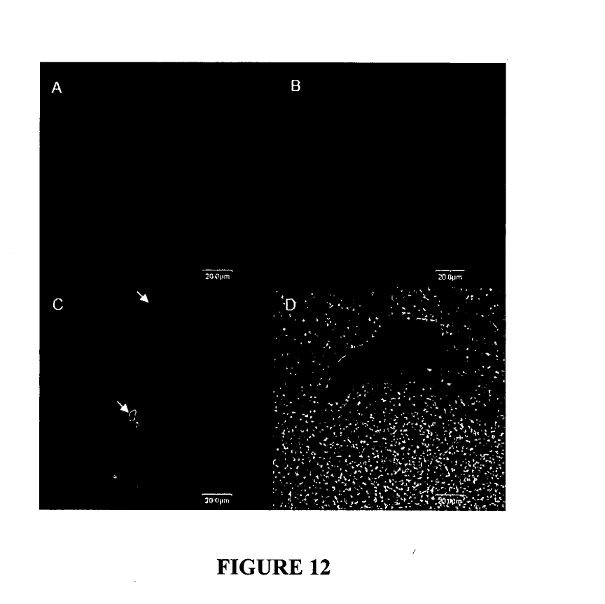

Our results (FIG. 12) demonstrate that the 2.2 kb hGFAP promoter is capable of directing HSC-specific expression in vivo by double staining the same transgenic liver tissue section with antibodies against GFAP and GFP respectively, and co-localizing on the same HSCs.

### Example 2

Materials and Methods

Cell Lines and Culture Conditions

The rat HSC-T6 cell line (Vogel et al. (2000), J Lipid Res. 41(6):882), a generous gift from Dr. Scott Friedman of the Mount Sinai School of Medicine, was previously transfected with a GFAP-LacZ reporter transgene, resulting in a stable line termed T6/GFAP-lacZ (Maubach et al., (2006) World J Gastroenterol. 12(5):723-30). All the cell culture media and reagents were purchased from Invitrogen (CA, USA). The cells were routinely cultured in Dulbecco's Modified Eagle Medium (DMEM), supplemented with 10% fetal bovine serum (FBS), 100 units of penicillin and 100 micrograms of streptomycin per milliliter at 37° C. in a humidified atmosphere of 5% CO₂. The cells were routinely split twice a week in a 1:10 ratio by trypsinization (0.05% trypsin/0.53 mM EDTA).

Cell Treatment with TGF-β1, PDGF-BB, EGCG and Genistein

EGCG (Cat. E4143) and genistein (Cat. G6649) were purchased from Sigma Chemicals (St. Louis, USA) and used without further purification. Stock solutions of compounds were prepared in DMSO. The final DMSO concentrations in the vehicle controls were kept between 0.1%-0.2%. Recombinant human TGF-β1 and PDGF-BB were purchased from BioVision (CA, USA). Stock solutions of cytokines were prepared in 1×PBS (pH7.4).

Cells were initially seeded in 12-well plates in DMEM supplemented with 10% FBS and weaned to a low serum DMEM (0.5% FBS) at a confluence of 70-80% for 12 hours of adaptation. Cells were then incubated with different concentrations of compounds for various amounts of time before being assayed for β-galactosidase activity and GFAP mRNA. For compound attenuation studies, cells were treated with a pro-fibrotic cytokine (TGF-β1 or PDGF-BB) either sequentially or concurrently with a compound (EGCG or genistein). In the sequential scheme, cells were first treated with a cytokine for 1-3 days, then the cytokine-containing culture medium was replaced with a fresh medium containing a particular compound only for various times. Details of the cytokine/compound treatments can be found in the individual figure legends.

X-Gal Staining and Quantification of β-Galactosidase Activity

A lacZ reporter assay kit for cell staining (InvivoGen, CA, USA) was used to detect the reporter gene activity in T6/GFAP-lacZ cells treated with EGCG or genistein. The staining results were documented with a digital camera (DP12) attached to an Olympus inverted bright field microscope (IX51) after development for 60 min. For quantification, the β-galactosidase activity in cell extracts was measured using a chemiluminescent assay kit (Roche, Mannheim, Germany) in a 96-well plate format, according to the manufacturer's instructions. Briefly, cells were washed twice with ice-cold 1×PBS (pH 7.4), lysed for 30 minutes at room temperature with the lysis buffer, and supernatant was collected for assays. Protein concentration was determined with a BCA protein assay kit (Pierce, Ill., USA). Specific β-galactosidase activity was obtained by normalizing against total cellular protein content.

Real-Time RT-PCR for Endogenous Rat GFAP mRNA

Total RNA was reverse-transcribed to cDNA using Taqman's reverse transcription reagent (Applied Biosystems, CA, USA). A total of 200 ng of RNA in 7.7 μl of nuclease-free water was added to 2 μl 10× reverse transcriptase buffer, 4.4 μl 25 mM magnesium chloride, 4 μl deoxyNTP mixtures, 1 μl random hexamers, 0.4 μl RNase inhibitor and 0.5 μl reverse transcriptase (50 U/μl) in a final reaction volume of 20 μl. The reaction was performed for 10 min at 25° C. (annealing), 30 min at 48° C. (cDNA synthesis) and 5 min at 95° C. (enzyme denaturation).

Real-time quantitative PCR was carried out with an ABI 7500 Fast Real-time PCR System (Applied Biosystems, CA, USA). Two micro liters of sample cDNA were used in each PCR reaction, with β-actin gene as a reference for normalization. The primers and probes for rat β-actin and GFAP were ordered from Taqman's assay-on-demand database. The PCR reaction was performed under a default profile consisting of 95° C. for 20 seconds (enzyme denaturation) and 40 cycles of amplification (denaturation 3 seconds, annealing and extension 30 seconds). Some of the real-time RT-PCR reactions were separated on an agarose gel for visual confirmation of discrete products (data not shown).

Relative quantitation of the target mRNA was calculated using the comparative threshold cycle (C_{T}) methods as described in the User Bulletin #2 (ABI Prism 7700 Sequence Detection System). C_{T} indicates the fractional cycle number at which the amount of amplified target reaches a fixed threshold within the linear phase of gene amplification. ΔC_{T}, which reflects the difference between C_{T target} and C_{T β-actin}, is inversely correlated to the abundance of mRNA transcripts in the samples. ACT for each sample was normalized against control experiment or calibrator and expressed as ΔΔC_{T}. Relative quantitation is given by 2^{-ΔΔCT} to express the up-regulation or down-regulation of the target gene under the treatments compared to the control.

Statistical Analysis

All quantitative results were presented as mean ±SE. Experimental data were analyzed using two-tailed Student's t-test assuming unequal variances. A P-value≦0.05 was considered statistically significant.

Results

EGCG and Genistein Inhibited HSC-T6 Cell Proliferation

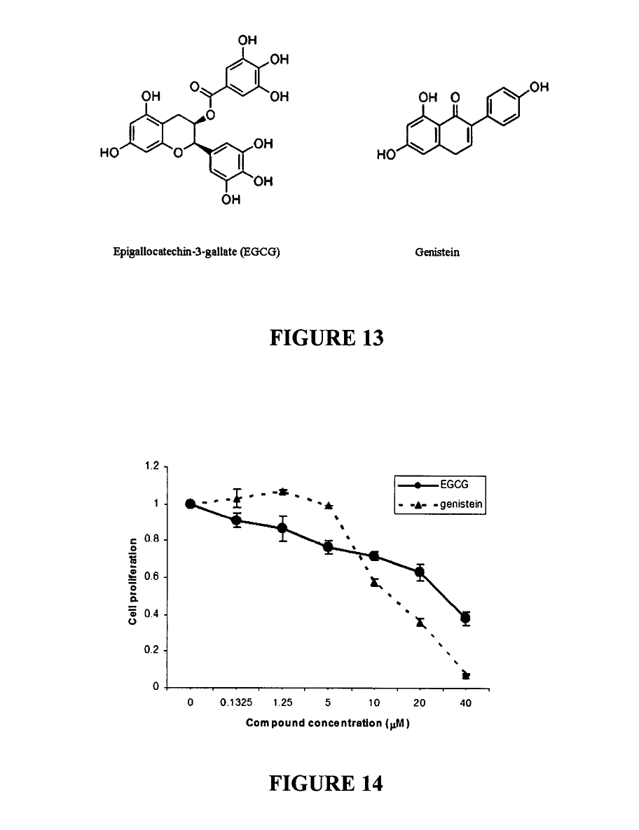

EGCG is a major catechin from green tea leaves associated with a wide range of beneficial activities including anti-oxidation, anti-inflammation and anti-fibrosis, whereas genistein is a major flavonoid found abundantly in soy bean with anti-tumor and pro-apoptotic activities. Their chemical structures are illustrated in FIG. 13. Several published reports have shown that these two compounds inhibited cell proliferation of HSC at different activation or transformation status (Chen et al., (2002) Biochem J 368(Pt 3):695-704; Chen and Zhang, (2003) J Biol Chem. 278:23381-9; Kang et al, (2001) Acta Pharmacol Sin. 22:793-6; Liu et al., (2002) World J Gastroenterol. 8:739-45). To ensure that the responsive characteristics of HSC were preserved in our transformed T6/GFAP-lacZ cells (Maubach et al., (2006) World J Gastroenterol. 12(5):723-30), the cells were treated with 25 μM of EGCG or 40 μM of genistein for 24 hours in a low serum (0.5% FBS) DMEM medium. Then the cells were fixed and stained with X-gal substrate for 60 min prior to photography.

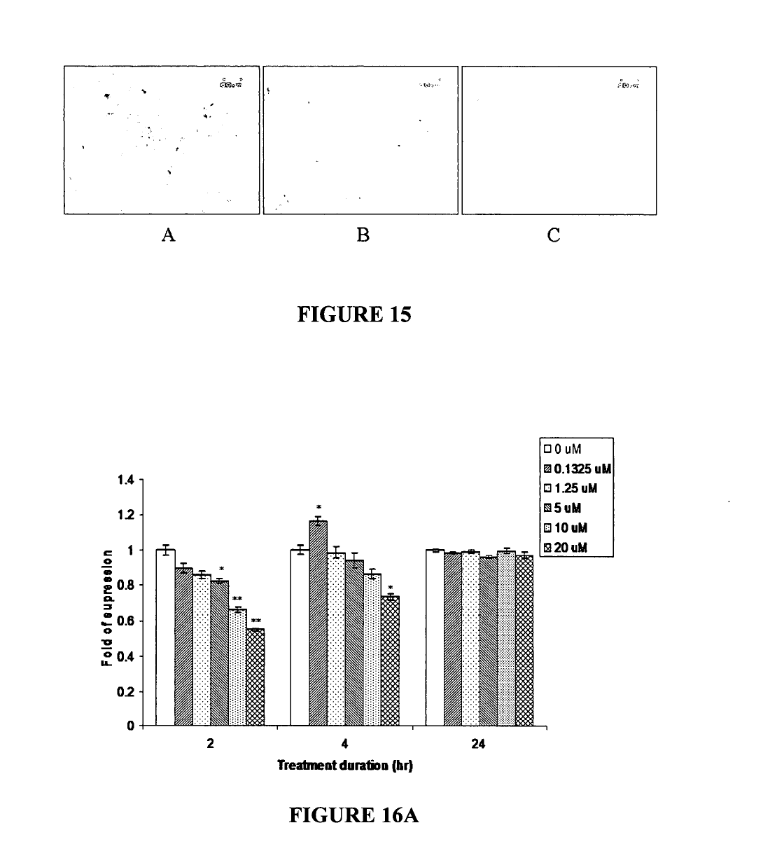

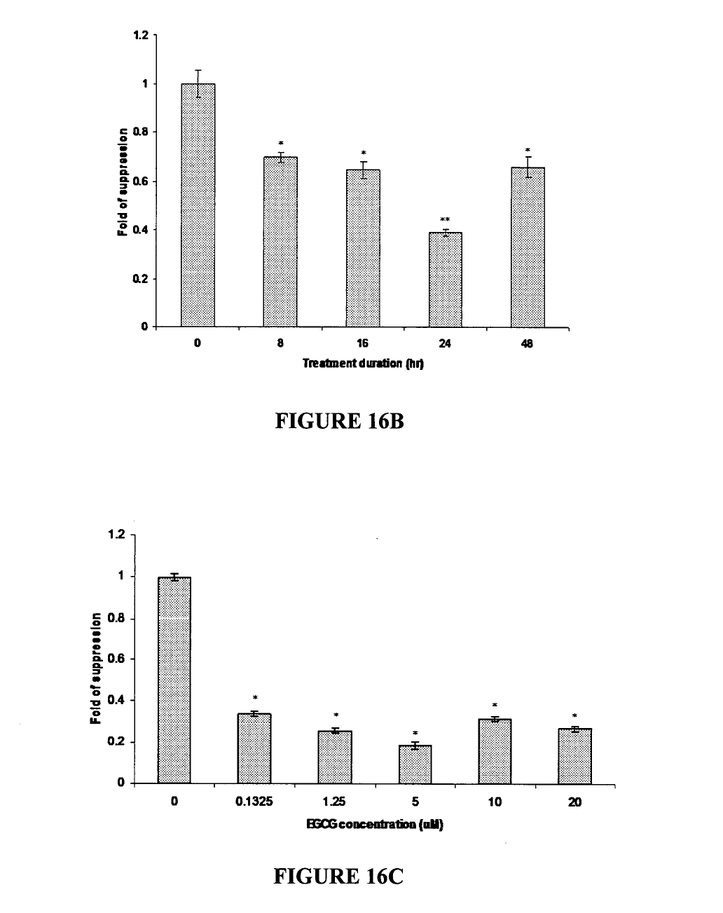

It was apparent that cells treated with EGCG (FIG. 15 Panel B) and genistein (FIG. 15 Panel C) displayed significant proliferation inhibition, as evident by fewer cells when compared to the control (FIG. 15 Panel A). The IC₅₀ values for the EGCG- and the genistein-mediated proliferation cell inhibition were determined to be 31 μM and 25 μM respectively in a low serum (0.5% FBS) medium (FIG. 14), by using the tetrazolium-based proliferation assay kit (Promega, Wis., USA). The EGCG IC₅₀ value obtained for the T6/GFAP-lacZ cells was in a comparable range to those reported for the activated (Sakata et al, (2004) J Hepatol. 40: 52-9) and immortalized human HSCs (Higashi et al, (2005) J Lab Clin Med. 145: 316-22), and for the primary rat HSCs (Chen et al., (2002) supra; Chen and Zhang, (2003) supra). However our genistein IC₅₀ value for the T6/GFAP-lacZ cells was three times lower than that for the T6 cells cultured in DMEM containing 10% FBS (Kang et al., (2001) supra). Our additional data also confirmed that no significant difference in IC₅₀ value was observed between the T6 and the T6/GFAP-lacZ cells (data not shown).

EGCG Suppressed GFAP-lacZ and Endogenous GFAP in a Dose- and Time-Dependent Manner

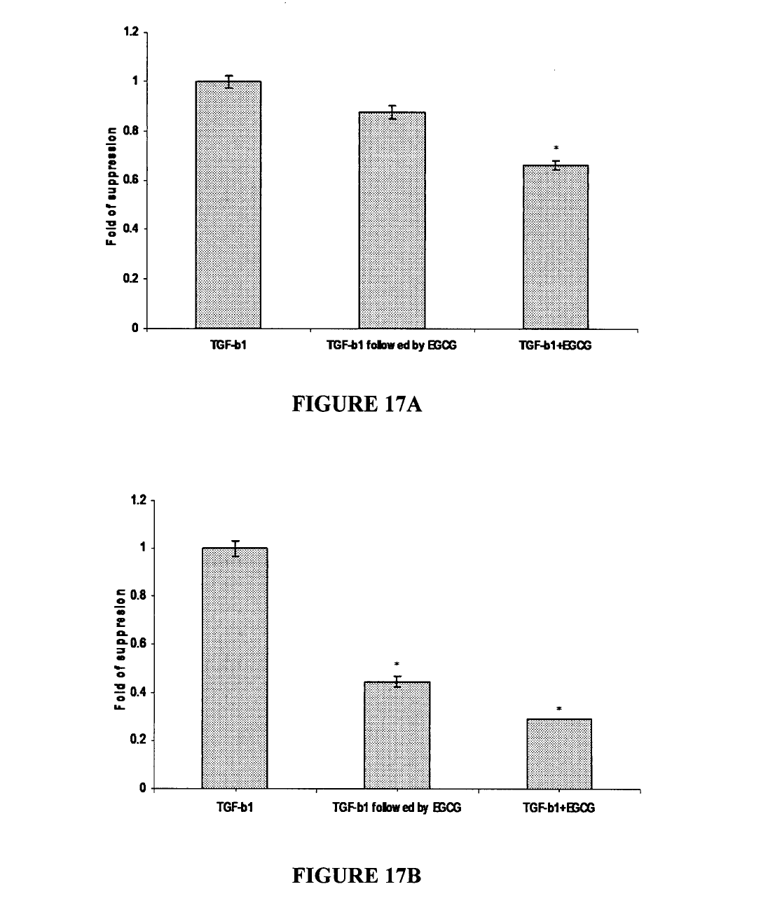

No background endogenous β-galactosidase activity was detected by using a chemiluminescent assay kit in the untransfected T6 cell extracts (data not shown). In contrast, significant β-galactosidase activity was reliably detected in the T6/GFAP-lacZ cells (FIG. 16A). Interestingly, a rapid and significant suppression on the transgene expression (representing 82% or 55% of the control respectively) was observed in cells treated with 5 μM or 20 μM of the EGCG for merely two hours. The initial suppression seemed to be transient by nature, since the only suppression (at 73% of the control level) observed at the 4-hour time point was caused by the treatment with 20 μM of EGCG, the highest concentration used in this particular experiment. When the treatment was prolonged to 24 hours, the EGCG inhibitory effect was completely abolished for all dosages. The mechanism for the observed phenomenon was not clear, but probably due to the rapid depletion of active EGCG metabolite(s) by cellular metabolism and/or cellular adaptation. It was reported that the EGCG half-life in human plasma was about 5 hours (Yang et al., (1998) Cancer Epidemiol Biomarkers Prev. 7:351-4).

Another interesting feature was that the maximum suppression of GFAP-lacZ transgene (55% of the control) by EGCG was observed after 2 hours of incubation (FIG. 16A), whereas the maximum suppression on the endogenous GFAP (39% of the control) was seen at 24 hours after the compound addition (FIG. 16B). Similarly, the expression of the transgene rebound back to the non-treated level earlier than that of the endogenous GFAP. An earlier response by the same transgene was also noted for the TGF-β1 induction (Maubach et al., (2006) supra).

The EGCG dosage effect on the endogenous GFAP transcription was also investigated. At 130 nM, a concentration significantly below the IC₅₀ value (31 μM), the GFAP transcription was significantly suppressed to 34% of the control level, suggesting the EGCG being a potent inhibitor of GFAP transcription in HSC (FIG. 16C). The minimum concentration of EGCG capable of causing significant inhibition of GFAP transcription was not systematically investigated in the current study. On the other hand, no further suppression was observed with increasing dosages up to 20 μM.

EGCG Attenuated Elevation of GFAP-lacZ and GFAP Induced by TGF-β1 or PDGF-BB

It was recently reported that the expression of endogenous and transgenic GFAP was induced by TGF-β1 in a dose- and time-dependent manner in the T6/GFAP-lacZ HSC line (Maubach et. al, (2006), supra). To investigate whether the cytokine-induced elevation in GFAP-lacZ and GFAP can be attenuated by EGCG, cells were subject to 1 or 10 ng/ml of TGF-β1 treatment either sequentially or concurrently with different concentrations of EGCG for various amounts of time prior to assaying for β-galactosidase activity and GFAP mRNA. For comparison purpose, assay values from cells treated with TGF-β1 or PDGF-BB alone were normalized to 1 for all attenuation experiments.

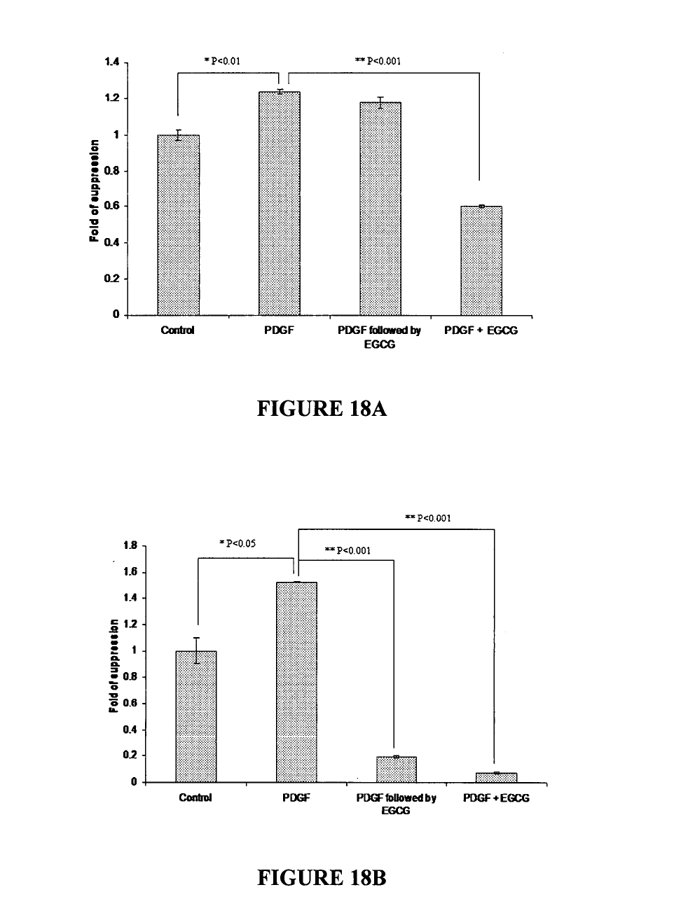

As shown in FIG. 17A, the β-galactosidase activity in cells treated with 10 ng/ml of TGF-β1 for 24 hours showed a trend of attenuation (87% of the control, but not statistically significant) after a sequential incubation with 20 μM of EGCG for additional 2 hours. However a significant suppression (66% of the control, P<0.005) was observed for the TGF-β1-treated cells concurrently incubated with 20 μM of EGCG for 26 hours (FIG. 17A). Similarly the endogenous GFAP mRNA levels were significantly suppressed down to 45% of the control (P<0.001) in the sequential treatment scheme (1 ng/ml of TGF-β1 for 48 hours, followed by 5 μM of EGCG for additional 8 hours) and to 29% of the control (P<0.001) in the concurrent scheme (1 ng/ml of TGF-β1 together with 5 μM of EGCG for a total of 56 hours), as shown in FIG. 17B.

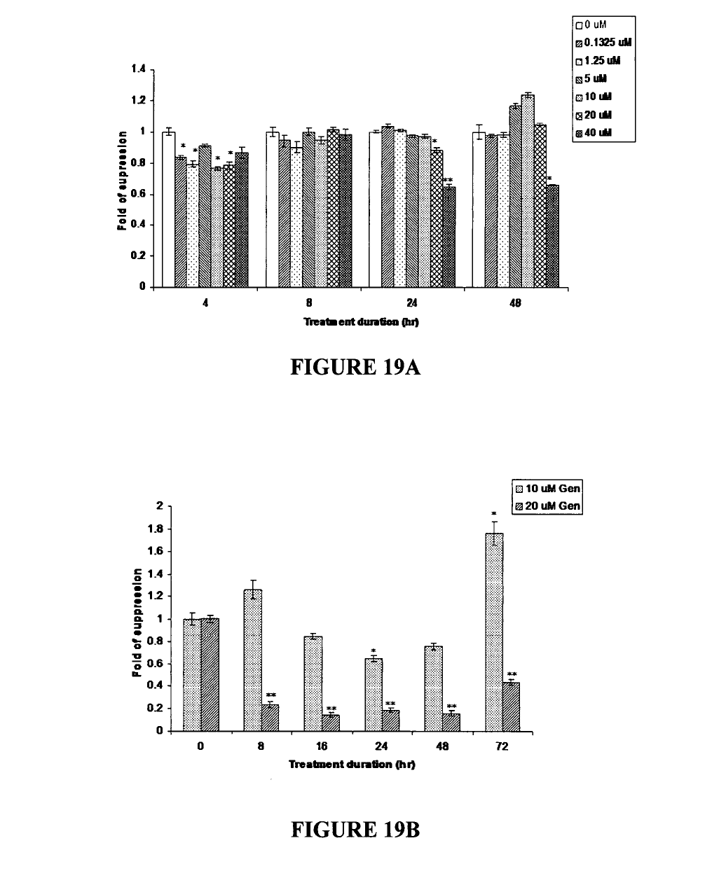

In addition to TGF-β1 induction (Maubach et al., (2006) supra), it was also shown here that another pro-fibrotic cytokine (i.e., 10 ng/ml of PDGF-BB) could induce the transgenic GFAP-lacZ expression by 24% (P<0.01) (FIG. 6A) and the endogenous GFAP by 53% (P<0.05) in the T6/GFAP-lacZ cells after being cultured for 48 and 24 hours respectively in a low serum DMEM. More importantly, the PDGF-induced GFAP elevation was greatly suppressed to 60% of the transgenic control (P<0.001; FIG. 18A) and 8% of the endogenous control (P<0.001; FIG. 18B) after incubation with 10 μM of EGCG for two days.

Genistein Suppressed GFAP-lacZ and Endogenous GFAP Expression in a Dose- and Time-Dependent Manner

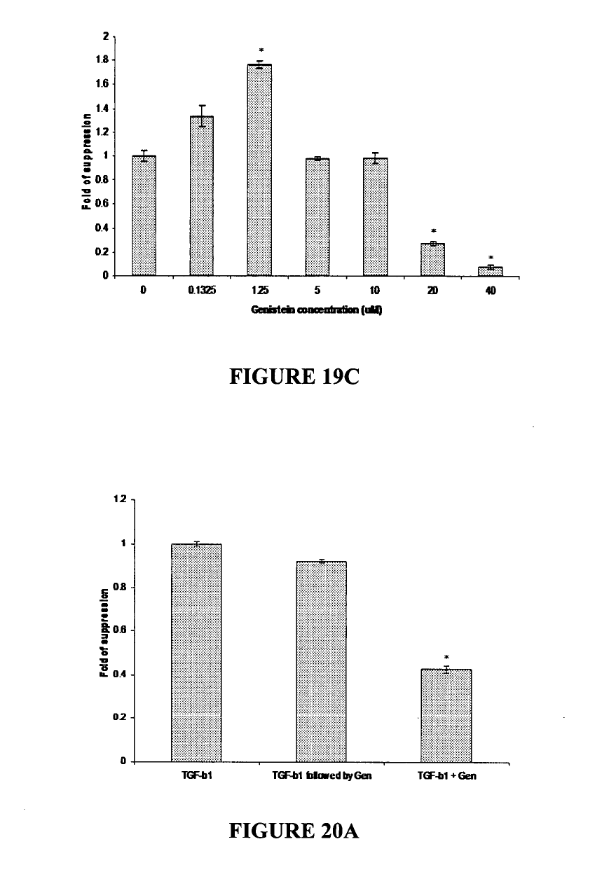

Compared to EGCG, fewer studies have been conducted to study the effects of genistein on HSCs. For example, genistein was shown to inhibit collagen synthesis and cell proliferation in rat HSCs (Kang et al., (2001) supra; Liu et al., (2002) supra). To investigate whether genistein would exert any influence on the GFAP signaling, the T6/GFAP-lacZ cells were treated with various concentrations of genistein and assayed after 4, 8, 24 and 48 hours of incubation. As shown in FIG. 19A, genistein inhibited the expression of the GFAP-lacZ transgene in a dose- and time-dependent manner. For comparison, the inhibiting power of genistein on GFAP-lacZ was less impressive than that of EGCG, nevertheless both compounds started to inhibit the transgene expression after only 2 hours of incubation (FIGS. 16A and 19A). The largest suppression (65% of the control) was seen for 40 μM of genistein at 24 hours (FIG. 19A).

For time-course study of the endogenous GFAP transcription, cells were treated with 10 or 20 μM of genistein and assayed for mRNA level at after 0, 8, 16, 24, 48, and 72 hours of incubation. A strong suppression (FIG. 19B) was observed for the cells treated with 20 μM of genistein starting from 8 hours (down to 23% of the control; P<0.001) and lasting to at least 72 hours (43% of the control; P<0.001). At a lower dose of genistein (10 μM), the effects were less predictable, ranging from suppression at 24 hours to induction at 72 hours. A follow-up dose experiment confirmed that the reliable inhibitory concentration of genistein should be higher than 10 μM, e.g., at the 20-40 μM range (FIG. 19C).

Genistein Attenuated Elevation of GFAP-lacZ and GFAP Induced by TGF-β1 or PDGF-BB

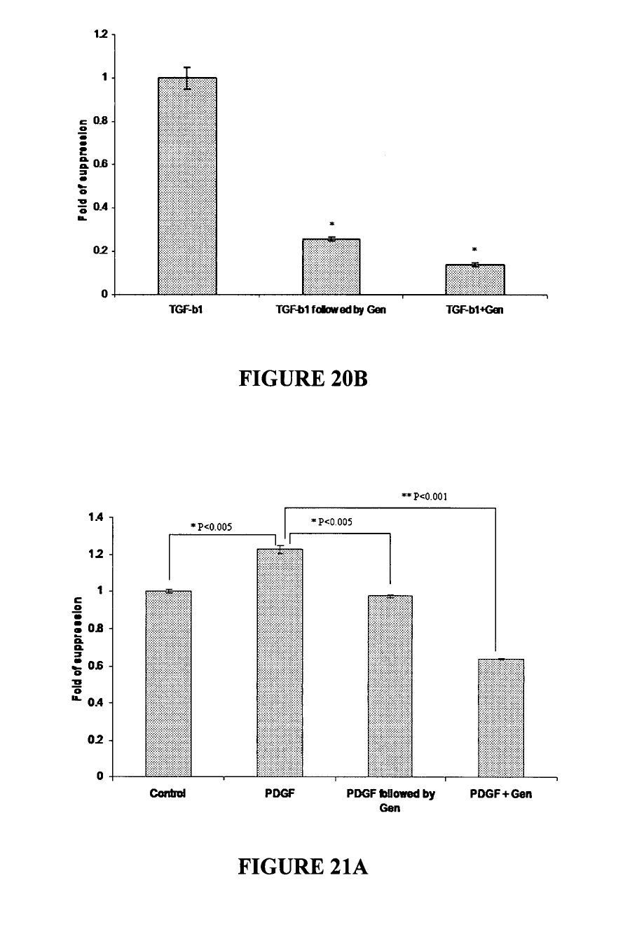

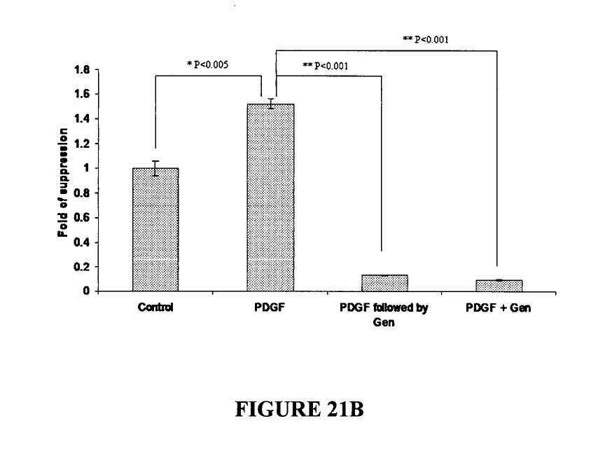

When 40 μM of genistein and 10 ng/ml of TGF-β1 were concurrently introduced into the G6/GFAP-lacZ cells for 48 hours, the transgenic β-galactosidase activity was decreased to 43% of the TGF-β1-alone control (P<0.005), whereas the suppressing effect was negligible in the sequential genistein treatment scheme (FIG. 20A). The TGF-β1-induced GFAP transcription was effectively blocked by 20 μM of genistein in either the sequential (26% of the control; P<0.001) or the concurrent (14% of the control; P<0.001) treatment scheme (FIG. 20B). Finally, 40 μM of genistein was also found to be very effective in attenuating the PDGF-BB-induced elevation in GFAP-lacZ to 79% (P<0.005) and 48% (P<0.001) of PDGF-BB-alone control in the sequential and the concurrent schemes respectively (FIG. 21A). For the endogenous GFAP, the transcription was inhibited even more dramatically by a lower concentration of genistein (20 μM) to 8.6% (P<0.001) and 6% (P<0.001) of the PDGF-BB-alone control in the sequential and the concurrent schemes respectively (FIG. 21B).

As can be understood by one skilled in the art, many modifications to the exemplary embodiments described herein are possible. The invention, rather, is intended to encompass all such modification within its scope, as defined by the claims.

All documents referred to herein are fully incorporated by reference.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. All technical and scientific terms used herein have the same meaning as commonly understood by one of ordinary skill in the art of this invention, unless defined otherwise.

What is claimed is:

1. A method for expressing a transgenic product in a hepatic stellate cell, the method comprising transfecting the hepatic stellate cell with a vector comprising a glial fibrillary acidic protein promoter operably coupled to a DNA sequence encoding the transgenic product, wherein the glial fibrillary acidic protein promoter consists of the sequence set forth in SEQ. ID NO. 1, or is an allelic variant of the sequence set forth in SEQ ID NO: 1.

2. The method according to claim 1 wherein the glial fibrillary acidic protein promoter sequence consists of the sequence set forth in SEQ ID NO: 1.

3. The method according to claim 1 wherein the transgenic product is a marker molecule.

4. The method according to claim 3 wherein the marker molecule is a marker protein.

5. The method according to claim 4 wherein the marker protein is β-galactosidase.

6. The method according to claim 1 wherein the transgenic product is a therapeutic molecule.

7. The method according to claim 6 wherein the therapeutic molecule is a therapeutic polypeptide.

8. The method according to claim 7 wherein the therapeutic polypeptide is Smad 7, a dominant negative allele of Smad 3, Smad 4 or a transforming growth factor receptor or a platelet derived growth factor receptor, or a diptheria toxin.

9. The method according to claim 7 wherein the therapeutic polypeptide is an anti-fibrotic polypeptide.

10. The method according to claim 9 wherein the anti-fibrotic polypeptide is interleukin-10.

11. The method according to claim 1 wherein the transgenic product is a small interfering RNA.

12. The method according to claim 11 wherein the small interfering RNA is complementary to a portion of a TGF-β1 mRNA or a portion of a platelet-derived growth factor mRNA.

13. The method according to claim 11 wherein the small interfering RNA is complementary to a portion of a mRNA encoding an extracellular matrix protein.

14. The method according to claim 13 wherein the extracellular matrix protein is collagen α1(I), integrin, laminin or fibronectin.

15. The method according to claim 1 wherein the hepatic stellate cell is a HSC-T6, LX-1 or LX-2.

16. The method according to claim 15 wherein the hepatic stellate cell is HSC-T6.

17. The method according to claim 15 wherein the hepatic stellate cell is LX-2.

18. An isolated transgenic hepatic stellate cell, the cell comprising a transgene operably coupled to a glial fibrillary acidic protein promoter, wherein the promoter consists of the sequence set forth in SEQ ID NO: 1, or a sequence that is an allelic variant of SEQ ID NO: 1.

19. The isolated transgenic hepatic stellate cell according to claim 18 wherein the glial fibrillary acidic protein promoter consists of the sequence set forth in SEQ ID NO: 1.

20. The isolated transgenic hepatic stellate cell according to claim 18 wherein the transgene encodes a marker molecule.

21. The isolated transgenic hepatic stellate cell according to claim 20 wherein the marker molecule is a marker protein.

22. The isolated transgenic hepatic stellate cell according to claim 21 wherein the marker protein is β-galactosidase.

23. The isolated transgenic hepatic stellate cell according to claim 18 wherein the transgene encodes a therapeutic molecule.

24. The isolated transgenic hepatic stellate cell according to claim 23 wherein the therapeutic molecule is a therapeutic polypeptide.

25. The isolated transgenic hepatic stellate cell according to claim 24 wherein the therapeutic polypeptide is Smad 7, a dominant negative allele of Smad 3, Smad 4 or a transforming growth factor receptor or a platelet derived growth factor receptor, or a diphtheria toxin.

26. The isolated transgenic hepatic stellate cell according to claim 24 wherein the therapeutic polypeptide is an anti-fibrotic polypeptide.

27. The isolated transgenic hepatic stellate cell according to claim 25 wherein the anti-fibrotic polypeptide is interleukin-10.

28. The isolated transgenic hepatic stellate cell according to claim 18 wherein the transgene encodes a small interfering RNA.

29. The isolated transgenic hepatic stellate cell according to claim 28 wherein the small interfering RNA is complementary to a portion of a TGF-β1 mRNA.

30. The isolated transgenic hepatic stellate cell according to claim 28 wherein the small interfering RNA is complementary to a portion of a collagen α1(I) mRNA.

31. A method of identifying a fibrogenesis modulating agent, the method comprising:
a) detecting a first expression level of a gene that is operably coupled to a glial fibrillary acidic protein promoter in an expression system in the absence of a test compound;
b) detecting a second expression level of the gene in the expression system in the presence of the test compound; and
c) comparing the first expression level and the second expression level,
whereby the first expression level greater than the second expression level indicates that the test compound is an anti-fibrotic agent and the first expression level less than the second expression level indicates that the test compound is a pro-fibrotic agent, and wherein the promoter consists of the sequence set forth in SEQ ID NO: 1, or a sequence that is an allelic variant of SEQ ID NO: 1.

32. The method of claim 31 wherein the expression system is a hepatic stellate cell.

33. The method of claim 32 wherein the hepatic stellate cell is an HSC-T6 cell.

34. The method of claim 32 wherein the gene is the endogenous glial fibrillary acidic protein gene.

35. The method of claim 32 wherein the hepatic stellate cell is a transgenic hepatic stellate cell and the gene is a transgene.

36. The method of claim 35 wherein the transgene encodes a marker protein.

37. The method of claim 36 wherein the marker protein is β-galactosidase.

38. The method of claim 32 wherein the test compound is a putative anti-fibrotic agent and a pro-fibrotic agent is administered to the hepatic stellate cell prior to said detecting a first expression level and said detecting a second expression level.

39. The method of claim 38 wherein the pro-fibrotic agent is TGF-β1, PDGF-BB or lipopolysaccharide.

40. The method of claim 31 wherein the test compound is a putative anti-fibrotic agent, the method further comprising comparing the effect of the test compound with the effect of a known anti-fibrotic agent.

41. The method of claim 40 wherein the known anti-fibrotic agent is epigallocatechin gallate, genistein or N-acetylcysteine.

42. The method of claim 31 wherein the test compound is a putative pro-fibrotic agent, the method further comprising comparing the effect of the test compound with the effect of a known pro-fibrotic agent.

43. The method of claim 42 wherein the known pro-fibrotic agent is TGF-β1, PDGF-BB or lipopolysaccharide.

44. A method of treating a hepatic fibrosis related disorder in a subject, the method comprising administering to the subject an effective amount of a vector, the vector comprising a DNA sequence encoding a therapeutic product operably coupled to a glial fibrillary acidic protein promoter, wherein the glial fibrillary acidic protein promoter consists of the sequence set forth in SEQ. ID NO. 1, or is an allelic variant of the sequence set forth in SEQ ID NO: 1.

45. The method of treating a hepatic fibrosis related disorder according to claim 42, wherein the glial fibrillary acidic protein promoter consists of the sequence set forth in SEQ. ID NO. 1.

46. The method according to claim 42 wherein the subject is a human subject.

47. A pharmaceutical preparation comprising a vector comprising a sequence encoding a therapeutic product operably coupled to a glial fibrillary acidic protein promoter, wherein the glial fibrillary acidic protein promoter consists of the sequence set forth in SEQ. ID NO. 1, or is an allelic variant of the sequence set forth in SEQ ID NO: 1 for treating a hepatic fibrosis related disorder and a physiological carrier.

48. The pharmaceutical preparation according to claim 41 wherein the glial fibrillary acidic protein promoter consists of the sequence set forth in SEQ. ID NO. 1.

49. A method of treating a hepatic fibrosis related disorder in a subject, the method comprising administering to the subject an effective amount of a transgenic HSC, wherein the transgenic HSC comprises a transgene encoding a therapeutic product, said transgene operably coupled to a glial fibrillary acidic protein promoter, and wherein said promoter consists of the sequence set forth in SEQ. ID NO. 1, or is an allelic variant of the sequence set forth in SEQ ID NO: 1.

50. A method of diagnosing the presence of hepatic fibrosis in a subject or determining the prognosis of a subject having or being likely to develop hepatic fibrosis, the method comprising:
a) detecting expression level of a gene that is operably coupled to a glial fibrillary acidic protein promoter in a hepatic stellate cell from the subject; and
b) comparing the expression level in the hepatic stellate cell from the subject with expression of a gene that is operably coupled to a glial fibrillary acidic protein promoter in a hepatic stellate cell that is not associated with hepatic fibrosis;
wherein the glial fibrillary acidic protein promoter consists of the sequence set forth in SEQ ID NO: 1, or a sequence that is an allelic variant of SEQ ID NO: 1.

51. A kit comprising a vector comprising a sequence encoding a therapeutic product operably coupled to a glial fibrillary acidic protein promoter, wherein the glial fibrillary acidic protein promoter consists of the sequence set forth in SEQ. ID NO. 1, or is an allelic variant of the sequence set forth in SEQ ID NO:1, and instructions for diagnosing or treating a hepatic fibrosis related disorder in a subject.
